(19) United States
(12) Patent Application Publication
(10) Pub. No.: US 2017/0305883 A1
(43) Pub. Date: Oct. 26, 2017

(54) Compositions and Methods for Inhibiting BMP

(71) Applicants: THE BRIGHAM AND WOMEN'S HOSPITAL, INC., Boston, MA (US); THE UNITED STATES OF AMERICA, AS REPRESENTED BY THE SECRETARY, DEPARTMENT OF HEALTH AND HUMAN SE.., Bethesda, MD (US); UNIVERSITY OF HOUSTON SYSTEM, Houston, TX (US)

(72) Inventors: Paul B. Yu, Boston, MA (US); Gregory D. Cuny, Houston, TX (US); Agustin H. Mohedas, Somerville, MA (US); Arthur Lee, Gaithersburg, MD (US)

(21) Appl. No.: 15/516,273

(22) PCT Filed: Oct. 1, 2015
(86) PCT No.: PCT/US2015/053545

§ 371 Date: Mar. 31, 2017

Related U.S. Application Data
(60) Provisional application No. 62/058,377, filed on Oct. 1, 2014.

(51) Int. Cl. C07D 401/14; C07D 409/04; C07D 403/02; C07D 401/04; C07D 221/22; C07D 213/02; A61K 31/551; C07D 417/14; A61K 31/44

(52) U.S. Cl. CPC C07D 401/14; C07D 417/14; C07D 409/04; C07D 403/02; A61K 31/44; C07D 221/22; C07D 213/02; A61K 31/551; C07D 401/04

(57) ABSTRACT

The present invention provides small molecule inhibitors of BMP signaling and compositions and methods for inhibiting BMP signaling. These compounds and compositions may be used to modulate cell growth, differentiation, proliferation, and apoptosis, and thus may be useful for treating diseases or conditions associated with BMP signaling, including inflammation, cardiovascular disease, hematological disease, cancer, and bone disorders, as well as for modulating cellular differentiation and/or proliferation. These compounds and compositions may also be used to reduce circulating levels of ApoB-100 or LDL and treat or prevent acquired or congenital hypercholesterolemia or hyperlipoproteinemia; diseases, disorders, or syndromes associated with defects in lipid absorption or metabolism; or diseases, disorders, or syndromes caused by hyperlipidemia.

### STATEMENT REGARDING FEDERALLY SPONSORED RESEARCH OR DEVELOPMENT

This invention was made with Government support under Grant Numbers HL079943 and AR057374, awarded by the National Institutes of Health and under project number 1ZIBTR000002 awarded by the National Center for Advancing Translational Sciences. The Government has certain rights in this invention.

### BACKGROUND OF THE INVENTION

Signaling involving the Transforming Growth Factor β (TGF-β) superfamily of ligands is central to a wide range of cellular processes, including cell growth, differentiation, and apoptosis. TGF-β signaling involves binding of a TGF-β ligand to a type II receptor (a serine/threonine kinase), which recruits and phosphorylates a type I receptor. The type I receptor then phosphorylates a receptor-regulated SMAD (R-SMAD; e.g., SMAD1, SMAD2, SMAD3, SMAD5, SMAD8 or SMAD9), which binds to SMAD4, and the SMAD complex then enters the nucleus where it plays a role in transcriptional regulation. The TGF superfamily of ligands includes two major branches, characterized by TGF-β/activin/nodal and Bone Morphogenetic Proteins (BMPs).

Signals mediated by bone morphogenetic protein (BMP) ligands serve diverse roles throughout the life of vertebrates. During embryogenesis, the dorsoventral axis is established by BMP signaling gradients formed by the coordinated expression of ligands, receptors, co-receptors, and soluble inhibitors (Massague et al. Nat. Rev Mol. Cell. Biol. 1:169-178, 2000). Excess BMP signaling causes ventralization, an expansion of ventral at the expense of dorsal structures, while diminished BMP signaling causes dorsalization, an expansion of dorsal at the expense of ventral structures (Nguyen et al. Dev. Biol. 199: 93-110, 1998; Furthauer et al. Dev. Biol. 214:181-196, 1999; Mintzer et al. Development 128:859-869, 2001; Schmid et al. Development 127:957-967, 2000). BMPs are key regulators of gastrulation, mesoderm induction, organogenesis, and endochondral bone formation, and regulate the fates of multipotent cell populations (Zhao, Genesis 35:43-56, 2003). BMP signals also play critical roles in physiology and disease, and are implicated in primary pulmonary hypertension, hereditary hemorrhagic telangiectasia syndrome, fibrodysplasia ossificans progressiva, and juvenile polyposis syndrome (Waite et al. Nat. Rev. Genet. 4:763-773, 2003; Papanikolaou et al. Nat. Genet. 36:77-82, 2004; Shore et al. Nat. Genet. 38:525-527, 2006).

The BMP signaling family is a diverse subset of the TGF-β superfamily (Sebald et al. Biol. Chem. 385:697-710, 2004). Over twenty known BMP ligands are recognized by three distinct type II (BMPRII, ActRIIa, and ActRIIb) and at least four type I (ALK1, ALK2, ALK3, and ALK6) receptors. Dimeric ligands facilitate assembly of receptor heteromers, allowing the constitutively-active type II receptor serine/threonine kinases to phosphorylate type I receptor serine/threonine kinases. Activated type I receptors phosphorylate BMP-responsive (BR-) SMAD effectors (SMADs 1, 5, and 8) to facilitate nuclear translocation in complex with SMAD4, a co-SMAD that also facilitates TGF signaling. In addition, BMP signals can activate intracellular effectors such as MAPK p38 in a SMAD-independent manner (Nohe et al. Cell Signal 16:291-299, 2004). Soluble BMP inhibitors, such as noggin, chordin, gremlin, and follistatin, limit BMP signaling by ligand sequestration.

A role for BMP signals in regulating expression of hepcidin, a peptide hormone and central regulator of systemic iron balance, has also been suggested (Pigeon et al. J. Biol. Chem. 276:7811-7819, 2001; Fraenkel et al. J. Clin. Invest. 115:1532-1541, 2005; Nicolas et al. Proc. Natl. Acad. Sci. U.S.A. 99:4596-4601, 2002; Nicolas et al. Nat. Genet. 34:97-101, 2003). Hepcidin binds and promotes degradation of ferroportin, the sole iron exporter in vertebrates. Loss of ferroportin activity prevents mobilization of iron to the bloodstream from intracellular stores in enterocytes, macrophages, and hepatocytes (Nemeth et al. Science 306:2090-2093, 2004). The link between BMP signaling and iron metabolism represents a potential target for therapeutics.

Given the tremendous structural diversity of the BMP and TGF-β superfamily at the level of ligands (>25 distinct ligands at present) and receptors (four type I and three type II receptors that recognize BMPs), and the heterotetrameric manner of receptor binding, traditional approaches for inhibiting BMP signals via soluble receptors, endogenous inhibitors, or neutralizing antibodies are not practical or effective. Endogenous inhibitors such as noggin and follistatin have limited specificity for ligand subclasses. Single receptors have limited affinity for ligand, whereas receptors heterotetramers exhibit more specificity for particular ligands. Neutralizing antibodies which are specific for particular ligands or receptors have been previously described, and are also limited by the structural diversity of this signaling system. Thus, there is a need in the art for pharmacologic agents that specifically antagonize BMP signaling pathways and that can be used to manipulate these pathways in therapeutic or experimental applications, such as those listed above.

### SUMMARY OF THE INVENTION

In one aspect, the invention provides compounds represented by general formula I or a pharmaceutically acceptable salt, ester, or prodrug thereof:

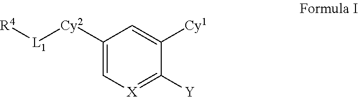

wherein

X is N;

Y is independently selected from hydrogen (such as protium, deuterium, or tritium), cyano, carboxyl, amino, monoalkylamino, dialkylamino, halo, alkyl (such as trifluoromethyl or other fluoroalkyl), or alkoxy;

Cy¹ is selected from substituted or unsubstituted aryl and heteroaryl;

Cy² is selected from a phenyl ring substituted with at least one non-protium (1H) substituent or a substituted or unsubstituted heteroaryl ring;

L₁ is absent or selected from substituted or unsubstituted alkyl and heteroalkyl;

R⁴ is selected from

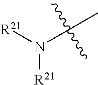

and a nitrogen-containing heterocyclyl or heteroaryl ring; and

R²¹, independently for each occurrence, is selected from H and substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfamoyl, or sulfonamide, preferably H or lower alkyl.

In certain embodiments, R⁴ is

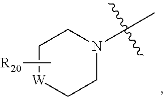

wherein

W is C(R²¹)₂, O, or NR²¹, preferably NR²¹, e.g., NH; and

R²⁰ is absent or represents from 1-6 substituents on the ring to which it is attached, preferably independently selected from substituted or unsubstituted alkyl, aralkyl, cycloalkyl, heterocyclyl, aryl, heteroaryl, heteroaralkyl, cycloalkylalkyl, heterocyclylalkyl, acyl, sulfonyl, sulfoxido, sulfamoyl, and sulfonamide, preferably absent.

In certain embodiments, Cy¹ is an aryl group substituted by 1 to 5 C₁-C₆ alkoxy groups, e.g., preferably substituted by alkoxy groups in the 3-, 4- and 5-positions relative to the bond to the ring bearing X.

In certain embodiments, Cy² is a substituted or unsubstituted nitrogen-containing heteroaryl group selected from pyridine, pyrazine, pyrimidine, oxazole, thiazole, and thiadiazole, e.g., selected from substituted or unsubstituted:

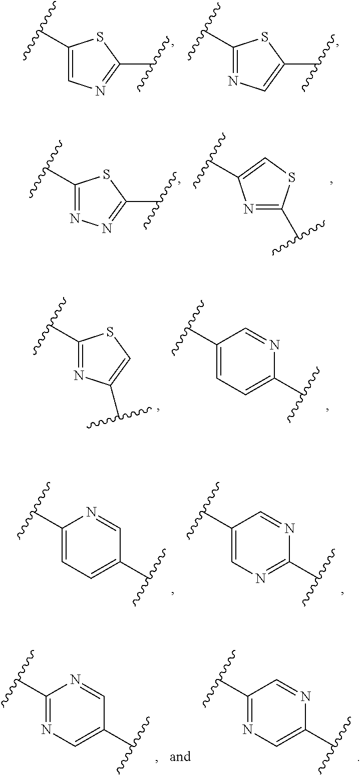

In certain embodiments wherein Cy² is substituted, the substituent is selected from deuterium, halogen (preferably fluoro or chloro), hydroxy, cyano, lower alkyl (preferably methyl or ethyl, most preferably methyl), or lower alkoxy (preferably methoxy).

In certain embodiments, Cy² is a phenyl ring. In certain such embodiments, Cy² is phenyl substituted with a non-protium substituent, either the substituent is halogen (preferably fluoro or chloro) or cyano, or is positioned ortho to L¹, or both.

In certain embodiments, Cy² is a 6-membered aryl or heteroaryil ring and L₁ is disposed on the meta- or para-position (preferably the para-position) of Cy² relative to the ring bearing X.

In certain preferred embodiments, L¹ is absent. In other embodiments, L₁ has a structure

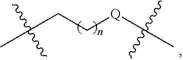

wherein Q is selected from CR¹⁰R¹¹, NR¹², O, S, S(O), and SO₂, R¹⁰ and R¹¹, independently for each occurrence, are selected from H and substituted or unsubstituted alkyl, cycloalkyl, heterocyclyl, cycloalkylalkyl, heterocyclylalkyl, amino, acylamino, carbamate, amino, amidino, cyano, sulfonyl, sulfoxido, sulfamoyl, or sulfonamido; R¹² selected from H and substituted or unsubstituted alkyl, cycloalkyl, heterocyclyl, heterocyclylalkyl, amino, acylamino, carbamate, amido, amidino, sulfonyl, sulfamoyl, or sulfonamide; and n is an integer from 0-4, wherein any CH₂ subunit of is optionally substituted with one or two lower alkyl groups, preferably one or two methyl groups.

In certain embodiments, R⁴ is

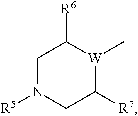

wherein W is N, CH, or CCH₃, preferably N or CH; R⁵ is selected from H and substituted or unsubstituted alkyl, acyl, or ester (thereby forming a carbamate); and R⁶ and R⁷ are each independently selected from H or alkyl, preferably from H or methyl, or R⁶ forms a one- or two-carbon (e.g., CH₂ or CH₂CH₂) bridge to the carbon atom adjacent to R⁷ and NR⁵.

In certain embodiments, Y is amino, monoalkylainino, or dialkylamino, preferably amino.

In yet another aspect, the invention provides compounds represented by general formula II or a pharmaceutically acceptable salt, ester or prodrug thereof:

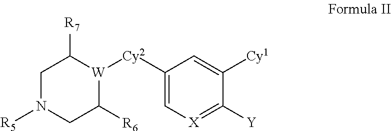

wherein

X is N;

Y is independently selected from hydrogen (such as protium, deuterium, or tritium), cyano, carboxyl, amino, monoalkylamino dialkylamino, halo, alkyl, or alkoxy;

Cy¹ is selected from substituted or unsubstituted aryl and heteroaryl;

Cy² is a substituted or unsubstituted aryl or heteroaryl ring;

W is N, CH, or CCH₃, preferably N or CH;

R⁵ is selected from H and substituted or unsubstituted alkyl, acyl, or ester (thereby forming a carbamate); and

R⁶ and R⁷ are each independently selected from H or alkyl, preferably from H or methyl, or R⁶ forms a one- or two-carbon (e.g., CH₂, or CH₂CH₂) bridge to the carbon atom adjacent to R⁷ and NR⁵.

In certain embodiments, R⁶ and R⁷ are both methyl, optionally disposed in a syn relationship to each other. In certain embodiments, R⁶ represents a one-carbon bridge, thereby forming a diazanorbornane bicycle, In certain such embodiments, W is N.

In certain embodiments, Cy¹ is an aryl group substituted by 1 to 5 C₁-C₆ alkoxy groups, e.g., preferably substituted by alkoxy groups in the 3-, 4- and 5-positions relative to the bond to the central pyridine ring.

In certain embodiments, Cy² is a substituted or unsubstituted nitrogen-containing heteroaryl group selected from pyridine, pyrazine, pyrimidine, oxazole, thiazole, and thiadlazole, e.g., selected from substituted or unsubstituted:

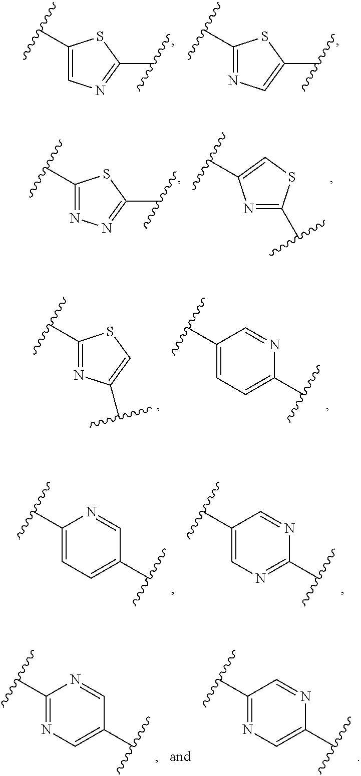

In certain embodiments wherein Cy² is substituted, the substituent is selected from deuterium, halogen (preferably fluoro or chloro), hydroxy, cyano, lower alkyl (preferably methyl or ethyl, most preferably methyl), or lower alkoxy (preferably methoxy).

In certain embodiments, Cy² is a, phenyl ring. In certain such embodiments, Cy² is phenyl substituted with a non-protium substituent, wherein the non-protium substituent is optionally selected from halogen (preferably fluoro or chloro) or cyano, or is positioned ortho to W, or both.

In certain embodiments, Cy² is a 6-membered aryl or heteroalyl ring and W is disposed on the meta- or para-position (preferably the para-position) of Cy² relative to the ring bearing X.

In certain embodiments, Y is amino, monoalkylamino, or dialkylamino, preferably amino.

In certain embodiments, the compound has a structure of one of compounds 10 and 13-33. In certain embodiments, the compounds of Formula I or II inhibit BMP-induced phosphorylation of SMAD1/5/8.

In one aspect, the invention provides a pharmaceutical composition comprising a compound as disclosed herein and a pharmaceutically acceptable excipient or solvent. In certain embodiments, a pharmaceutical composition may comprise a prodrug of a compound as disclosed herein.

In another aspect, the invention provides a method of inhibiting BMP-induced phosphorylation of SMAD1/5/8, comprising contacting a cell with a compound or composition as disclosed herein.

In certain embodiments, the method treats or prevents a disease or condition in a subject that would benefit by inhibition of Bone Morphogenetic Protein (BMP) signaling. In certain embodiments, the disease or condition is selected from pulmonary hypertension, hereditary hemorrhagic telangiectasia syndrome, cardiac valvular malformations, cardiac structural malformations, fibrodysplasia ossificans progressiva, juvenile familial polyposis syndrome, parathyroid disease, cancer (e.g., breast carcinoma, prostate carcinoma, renal cell carcinoma, bone metastasis, lung metastasis, osteosarcoma, and multiple myeloma), anemia, vascular calcification, atherosclerosis, valve calcification, renal osteodystrophy, inflammatory disorders (e.g., ankylosing spondylitis), infections with viruses, bacteria, fungi, tuberculosis, and parasites.

In certain embodiments, the method reduces the circulating levels of ApoB-100 and/or LDL and/or total cholesterol in a subject that has levels of ApoB-100 and/or LDL and/or total cholesterol that are abnormally high or that increase a patient's risk of developing a disease or unwanted medical condition. In certain embodiments, the method of reducing circulating levels of ApoB-100 and/or LDL and/or total cholesterol in a subject reduces the risk of primary or secondary cardiovascular events. In certain embodiments, the method treats or prevents a disease or condition in a subject that would benefit by inhibition of Bone Morphogenetic Protein (BMP) signaling. In certain embodiments, the disease or condition is selected from pulmonary hypertension; hereditary hemorrhagic telangiectasia syndrome; cardiac valvular malformations; cardiac structural malformations; fibrodysplasia ossificans progressive; juvenile familial polyposis syndrome; parathyroid disease; cancer (e.g., breast carcinoma, diffuse intrinsic pontine gliomas (DIPG), prostate carcinoma, renal cell carcinoma, bone metastasis, lung metastasis, osteosarcoma, and multiple myeloma); anemia; vascular calcification; vascular inflammation; atherosclerosis; acquired or congenital hypercholesterolemia or hyperlipoproteinemia; diseases, disorders, or syndromes associated with defects in lipid absorption or metabolism; diseases, disorders, or syndromes caused by hyperlipidemia; valve calcification; renal osteodystrophy; inflammatory disorders (e.g., ankylosing spondylitis); infections with viruses; bacteria; fungi; tuberculosis; and parasites.

In another aspect, the invention provides a method of treating hypercholesterolemia, hyperlipidemia, hyperlipoproteinemia or hepatic steatosis in a subject comprising administering an effective amount of a compound as disclosed herein. In certain such embodiments, the congenital hypercholesterolemia, hyperlipidemia, or hyperlipoproteinemia is autosomal dominant hypercholesterolemia (ADH), familial hypercholesterolemia (FH), polygenic hypercholesterolemia, familial combined hyperlipidemia (FCHL), hyperapobetalipoproteinemia, or small dense LDL syndrome (LDL phenotype B). In certain such embodiments, the hypercholesterolemia, hyperlipidemia, hyperlipoproteinemia or hepatic steatosis is acquired hypercholesterolemia, hyperlipidemia, hyperlipoproteinemia or hepatic steatosis. In certain such embodiments, the hypercholesterolemia, hyperlipidemia, hyperlipoproteinemia, or hepatic steatosis is associated with diabetes mellitus, hyperlipidemic diet and/or sedentary lifestyle, obesity, metabolic syndrome, intrinsic or secondary liver disease, biliary cirrhosis or other bile stasis disorders, alcoholism, pancreatitis, nephrotic syndrome, endstage renal disease, hypothyroidism, iatrogenesis due to administration of thiazides, beta-blockers, retinoids, highly active antiretroviral agents, estrogen, progestins, or glucocorticoids.

In another aspect, the invention provides a method of preventing cardiovascular disease in a subject with elevated markers of cardiovascular risk, comprising administering an effective amount of a compound as disclosed herein.

In another aspect, the invention provides a method of preventing and treating hepatic dysfunction in a subject associated with nonalcoholic fatty liver disease (NAFLD), steatosis-induced liver injury, fibrosis, cirrhosis, or non-alcoholic steatohepatitis (NASH) in a subject comprising administering an effective amount of a compound as disclosed herein.

In another aspect, the invention provides a method of inducing expansion or differentiation of a cell, comprising contacting the cell with a compound as disclosed herein. In certain embodiments, the cell is selected from an embryonic stem cell and an adult stem cell. In certain embodiments, the cell is in vitro.

In certain embodiments, a method of the invention may comprise contacting a cell with a prodrug of a compound as disclosed herein.

### BRIEF DESCRIPTION OF THE FIGURES

FIGS. 1a and 1b show the in vitro thermal shift kinase assay using the BMP and TGF-β type I receptors ALK2 (FIG. 1a ) and ALK5 (FIG. 1b ), respectively. A strong negative log-linear correlation is seen between thermal shift and biochemical IC50 for both (a) BMP (ALK2) and (b) TGF-β (ALK5) type 1 receptors.

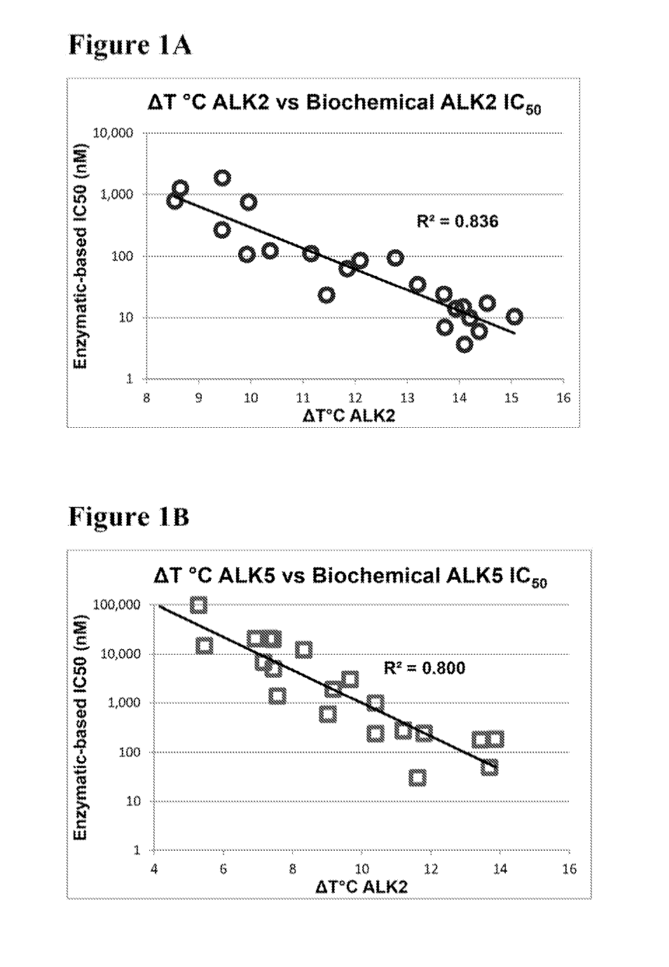

FIG. 2 shows the inhibition of constitutively active BMP (ALK1, ALK2, ALK3) and TGF-β (ALK4 and ALK5) type 1 receptors by compound 15 in cell-based luciferase reporter assay. Data shown are representative of more than 3 independent experiments, with data plotted as mean±S.E.M. (n=3 replicates).

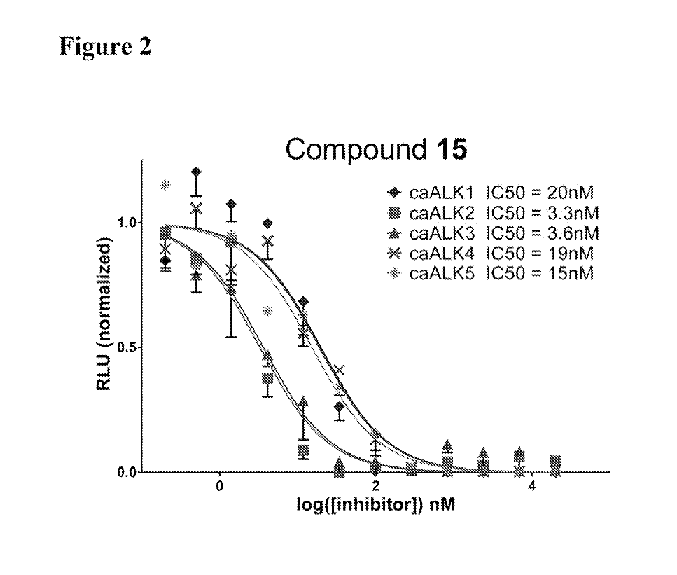

FIGS. 3a and 3b show the correlation between thermal shift of type 1 receptors and their corresponding cell-based IC₅₀.

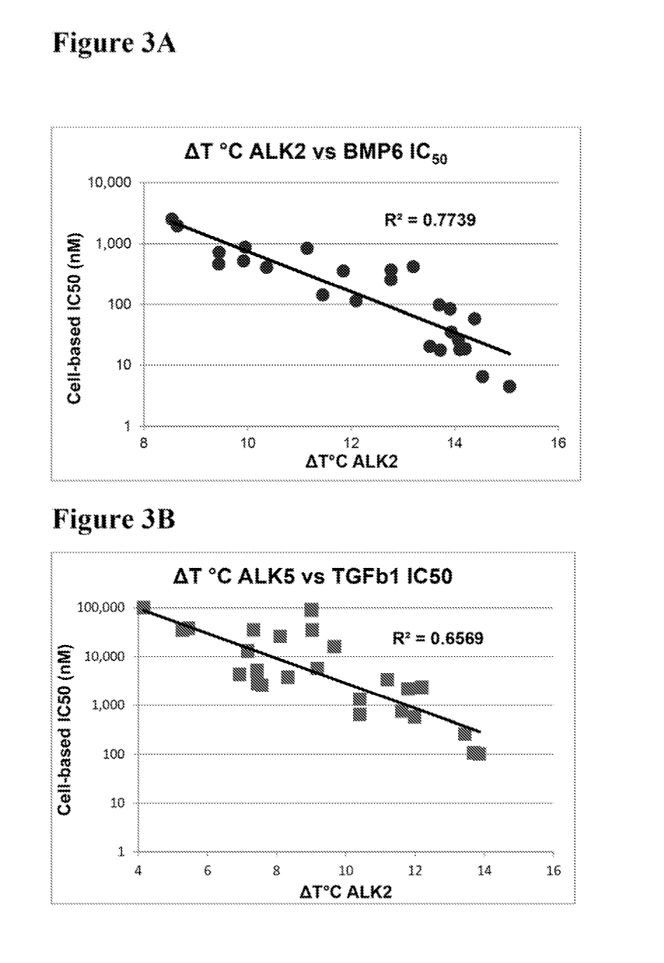

FIGS. 4a-d show the correlation of thermal shift and cell-based BMP/TGF-β inhibition assays of certain compounds of the invention. K02288 and compounds 11-15 are shown in FIG. 4a. Compounds 15-23 are shown in FIG. 4b. Compounds 10, 15, and 24-28 are shown in FIG. 4c. Compounds 29-33 are shown in FIG. 4d.

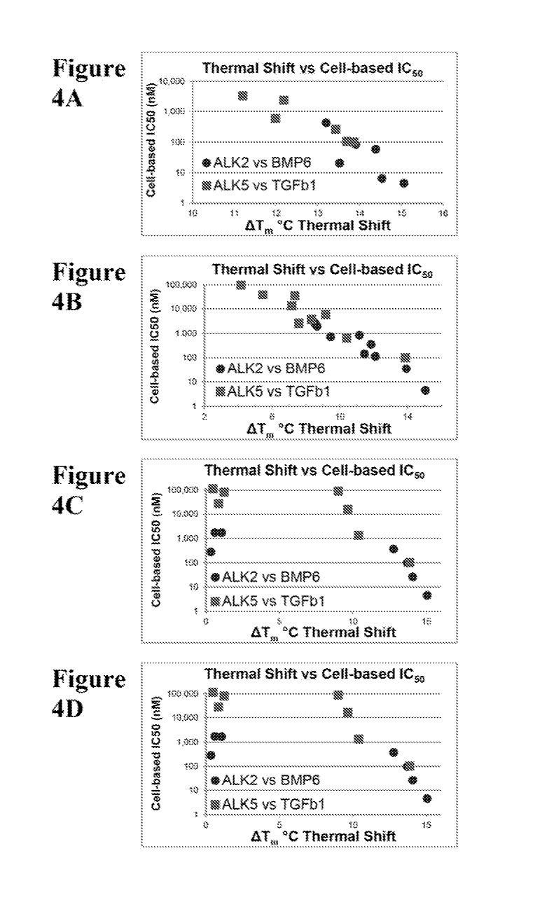

FIGS. 5a and 5b show the kinome dendrogram plot for compound 15 (FIG. 5a ) and compound 10 (FIG. 5b ).

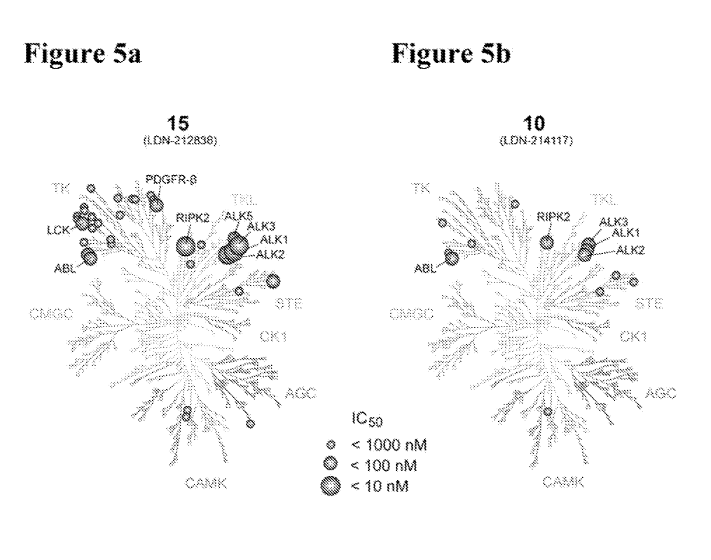

FIGS. 6a and 6b show the plots of cell based BMP (FIG. 6a ) and TGF-β (FIG. 6b ) IC₅₀ versus cell viability.

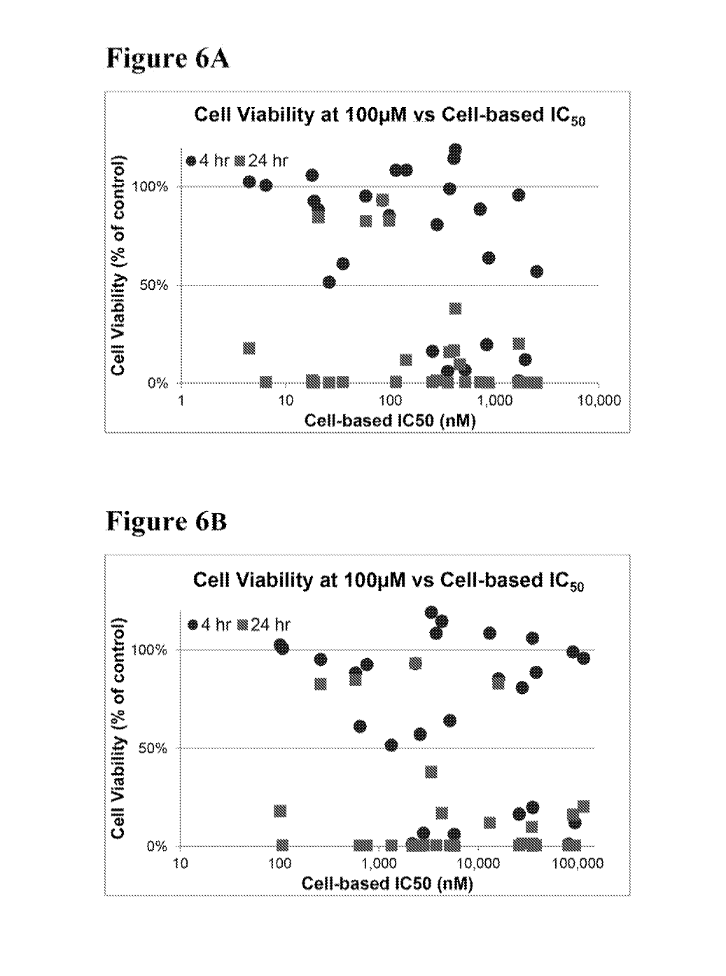

### DETAILED DESCRIPTION OF THE INVENTION

The invention provides for compounds that inhibit the BMP signaling pathway, as well as methods to treat or prevent a disease or condition in a subject that would benefit by inhibition of BMP signaling.

### I. Compounds

Compounds of the invention include compounds of Formula I or II as disclosed above and their salts (including pharmaceutically acceptable salts). Such compounds are suitable for the compositions and methods disclosed herein.

### II. Definitions

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—, preferably alkylC(O)NH—.

The term “acyloxy” is art-recognized and refers to a group represented by the general formula hydrocarbylC(O)O—, preferably alkylC(O)O—.

The term “aliphatic”, as used herein, includes straight, chained, branched or cyclic hydrocarbons which are completely saturated or contain one or more units of unsaturation. Aliphatic groups may be substituted or unsubstituted.

The term “alkoxy” refers to an oxygen having an alkyl group attached thereto. Representative alkoxy groups include methoxy, ethoxy, propoxy, tert-butoxy and the like.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated. In preferred embodiments, a straight chain or branched chain alkenyl has 1-12 carbons in its backbone, preferably 1-8 carbons in its backbone, and more preferably 1-6 carbons in its backbone. Exemplary alkenyl groups include allyl, propenyl, butenyl, 2-methyl-2-butenyl, and the like.

The term “alkyl” refers to the radical of saturated aliphatic groups, including straight-chain alkyl groups, and branched-chain alkyl groups. In preferred embodiments, a straight chain or branched chain alkyl has 30 or fewer carbon atoms in its backbone (e.g., C₁-C₃₀ for straight chains, C₃-C₃₀ for branched chains), and more preferably 20 or fewer. In certain embodiments, alkyl groups are lower alkyl groups, e.g. methyl, ethyl, n-propyl, i-propyl, n-butyl and n-pentyl.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. In certain embodiments, a straight chain or branched chain alkyl has 30 or fewer carbon atoms in its backbone (e.g., C₁-C₃₀ for straight chains, C₃-C₃₀ for branched chains). In preferred embodiments, the chain has ten or fewer carbon (C₁-C₁₀) atoms in its backbone. In other embodiments, the chain has six or fewer carbon (C₁-C₆) atoms in its backbone.

Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aryl or heteroaryl moiety.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated. In preferred embodiments, an alkynyl has 1-12 carbons in its backbone, preferably 1-8 carbons in its backbone, and more preferably 1-6 carbons in its backbone. Exemplary alkynyl groups include propynyl, butynyl, 3-methylpent-1-ynyl, and the like.

The term “amide”, as used herein, refers to a group

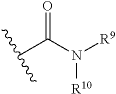

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

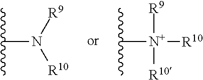

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with one or more aryl groups.

The term “aryl”, as used herein, include substituted or unsubstituted single-ring aromatic groups in which each atom of the ring is carbon. Preferably the ring is a 5- to 7-membered ring, more preferably a 6-membered ring. Aryl groups include phenyl, phenol, aniline, and the like.

The term “carbamate” is art-recognized and refers to a group

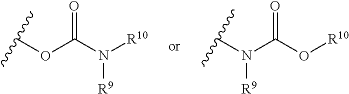

wherein R⁹ and R¹⁰ independently represent hydrogen or a hydrocarbyl group, such as an alkyl group.

The terms “carbocycle”, “carbocyclyl”, and “carbocyclic”, as used herein, refers to a non-aromatic saturated or unsaturated ring in which each atom of the ring is carbon. Preferably a carbocycle ring contains from 3 to 10 atoms, more preferably from 5 to 7 atoms.

The term “carbocyclylalkyl”, as used herein, refers to an alkyl group substituted with a carbocycle group.

The term “carbonate” is art-recognized and refers to a group —OCO₂—R⁹, wherein R⁹ represents a hydrocarbyl group, such as an alkyl group.

The term “carboxy”, as used herein, refers to a group represented by the formula —CO₂H.

The term “cycloalkyl”, as used herein, refers to the radical of a saturated aliphatic ring. In preferred embodiments, cycloalkyls have from 3-10 carbon atoms in their ring structure, and more preferably from 5-7 carbon atoms in the ring structure. Suitable cycloalkyls include cycloheptyl, cyclohexyl, cyclopentyl, cyclobutyl and cyclopropyl.

The term “ester”, as used herein, refers to a group —C(O)OR⁹ wherein R⁹ represents a hydrocarbyl group, such as an alkyl group or an aralkyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen”, as used herein, means halogen and includes chloro, fluoro, bromo, and iodo.

The term “heteroalkyl”, as used herein, refers to a saturated or unsaturated chain of carbon atoms including at least one heteroatom (e.g., O, S, or NR⁵⁰, such as where R⁵⁰ is H or lower alkyl), wherein no two heteroatoms are adjacent.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom (e.g., O, N, or S), preferably one to four or one to 3 heteroatoms, more preferably one or two heteroatoms. When two or more heteroatoms are present in a heteroaryl ring, they may be the same or different. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Preferred polycyclic ring systems have two cyclic rings in which both of the rings are aromatic. Heteroaryl groups include, for example, pyrrole, furan, thiophene, imidazole, oxazole, thiazole, pyrazole, pyridine, pyrazine, pyridazine, quinoline, and pyrimidine, and the like.

The term “heteroatom”, as used herein, means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, preferably six or fewer. A “lower alkyl”, for example, refers to an alkyl group that contains ten or fewer carbon atoms, preferably six or fewer. Examples of straight chain or branched chain lower alkyl include methyl, ethyl, isopropyl, propyl, butyl, tertiary-butyl, and the like. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitation aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Preferred polycycles have 2-3 rings. Each of the rings of the polycycle can be substituted or unsubstituted. In certain embodiments, each ring of the polycycle contains from 3 to 10 atoms in the ring, preferably from 5 to 7.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of the invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, an alkylthio, an acyloxy, a phosphoryl, a phosphate, a phosphonate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt or ester thereof.

The term “sulfonamide” is art-recognized and refers to the group represented by the general formulae

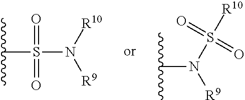

wherein R⁹ and R¹⁰ independently represents hydrogen or hydrocarbyl, such as alkyl.

The term “sulfoxide” is art-recognized and refers to the group —S(O)—R⁹, wherein R⁹ represents a hydrocarbyl, such as alkyl, aryl, or heteroaryl.

The term “sulfonate” is an-recognized and refers to the group —SO₃H, or a pharmaceutically acceptable salt or ester thereof.

The term “sulfone” is art-recognized and refers to the group —S(O)₂—R⁹, wherein R⁹ represents a hydrocarbyl, such as alkyl, aryl, or heteroaryl.

The term “thioester”, as used herein, refers to a group —C(O)SR⁹ or —SC(O)R⁹ wherein R⁹ represents a hydrocarbyl, such as alkyl.

The term “thioether”, as used herein, is equivalent to an ether, wherein the oxygen is replaced with a sulfur.

The term “urea” is art-recognized and may be represented by the general formula

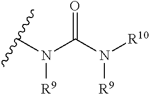

wherein R⁹ and R¹⁰ independently represent hydrogen or a hydrocarbyl, such as alkyl.

At various places in the present specification substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, propyl, isopropyl, n-butyl, sec-butyl, isobutyl, etc.

For a number qualified by the term “about”, a variance of 2%, 5%, 10% or even 20% is within the ambit of the qualified number

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “prodrug” is intended to encompass compounds which, under physiologic conditions, are converted into the therapeutically active agents of the present invention (e.g., a compound of Formula I or Formula II). A common method for making a prodrug is to include one or more selected moieties which are hydrolyzed under physiologic conditions to reveal the desired molecule. In other embodiments, the prodrug is converted by an enzymatic activity of the host animal. For example, esters (e.g., esters of alcohols or carboxylic acids) are preferred prodrugs of the present invention. In various embodiments disclosed herein (e.g., the various compounds, compositions, and methods), some or all of the compounds of formula A, compounds of any one of Formula I or Formula II, all or a portion of a compound of Formula I or Formula II in a formulation represented above can be replaced with a suitable prodrug, e.g., wherein a hydroxyl or carboxylic acid present in the parent compound is presented as an ester.

As used herein, the term “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “small molecule” refers to an organic molecule having a molecular weight less than about 2500 amu, preferably less than about 2000 amu, even more preferably less than about 1500 amu, still more preferably less than about 1000 amu, or most preferably less than about 750 amu. Preferably a small molecule contains one or more heteroatoms.

The phrase “activity of ALK2” means ALK-2 enzymatic activity (e.g., such as kinase activity; the ability of ALK-2 to phosphorylate BMP-responsive SMAD proteins) and/or ALK-2-mediated signaling (e.g., such as the ability of ALK-2 to mediate downstream signal transduction and transcriptional activity following activation of ALK-2 by binding of BMP ligands). In some embodiments, “activity of ALK2” means ALK2-mediated BMP signaling. In some embodiments, “activity of ALK2” means ALK2-mediated BMP-responsive gene transcription (e.g., transcriptional activity mediated by BMP/ALK2 signal transduction).

The phrase “activity of ALK5” means ALK-5 enzymatic activity (e.g., such as kinase activity; the ability of ALK-5 to phosphorylate TGF-β responsive SMAD proteins; the ability of ALK-5 to phosphorylate SMAD2 or SMAD3) and/or ALK-5-mediated signaling (e.g., such as the ability of ALK-5 to mediate downstream signal transduction and transcriptional activity following activation of ALK-5 by binding of TGF-β ligands). In some embodiments, “activity of ALK5” means ALK5-mediated TGF-β signaling. In some embodiments, “activity of ALK” means ALK5-mediated TGF-β-responsive gene transcription (e.g., transcriptional activity mediated by TGFβ/ALK5 signal transduction).

The phrase “activity of ALK1” means ALK-1 enzymatic activity (e.g., such as kinase activity; the ability of ALK-1 to phosphorylate BMP-responsive SMAD proteins) and/or ALK-1-mediated signaling (e.g., such as the ability of ALK-1 to mediate downstream signal transduction and transcriptional activity following activation of ALK-1 by binding of BMP ligands). In some embodiments, “activity of ALK1” means ALK1-mediated BMP signaling. In some embodiments, “activity of ALK1” means ALK1-mediated BMP-responsive gene transcription (e.g., transcriptional activity mediated by BMP/ALK1 signal transduction).

The phrase “activity of ALK3” means ALK-3 enzymatic activity (e.g., such as kinase activity; the ability of ALK-3 to phosphorylate BMP-responsive SMAD proteins) and/or ALK-3-mediated signaling (e.g., such as the ability of ALK-3 to mediate downstream signal transduction and transcriptional activity following activation of ALK-3 by binding of BMP ligands). In some embodiments, “activity of ALK3” means ALK3-mediated BMP signaling. In some embodiments, “activity of ALK3” means ALK3-mediated BMP-responsive gene transcription (e.g., transcriptional activity mediated by BMP/ALK3 signal transduction).

The phrase “activity of ALK4” means ALK-4 enzymatic activity (e.g., such as kinase activity; the ability of ALK-4 to phosphorylate activin-responsive SMAD proteins; the ability of ALK-4 to phosphorylate SMAD 2 or SMAD 3) and/or ALK-4-mediated signaling (e.g., such as the ability of ALK-4 to mediate downstream signal transduction and transcriptional activity following activation of ALK-4 by binding of activin ligands). In some embodiments, “activity of ALK4” means ALK4-mediated activin signaling. In some embodiments, “activity of ALK4” means ALK4-mediated activin-responsive gene transcription (e.g., transcriptional activity mediated by activin/ALK4 signal transduction).

The phrase “activity of ALK6” means ALK-6 enzymatic activity (e.g., such as kinase activity; the ability of ALK-6 to phosphorylate BMP-responsive SMAD proteins) and/or ALK-6-mediated signaling (e.g., such as the ability of ALK-6 to mediate downstream signal transduction and transcriptional activity following activation of ALK-6 by binding of BMP ligands). In some embodiments, “activity of ALK6” means ALK6-mediated BMP signaling. In some embodiments, “activity of ALK6” means ALK6-mediated GDF5 signaling. In some embodiments, “activity of ALK6” means ALK6-mediated BMP-responsive gene transcription (e.g., transcriptional activity mediated by BMP/ALK6 signal transduction).

Human ALK2 is a 509 amino acid protein. The protein sequence is published, for example, as GenBank accession number NP_001104537.1, (with corresponding nucleotide sequence at NM_001111067.2) UniProt entry Q04771.

Human ALK5 has, at least, two isoforms: a 503 amino acid protein (isoform 1) and a 426 amino acid protein. The protein sequence for human ALK5 isoform 1 is published, for example, as GenBank accession number NP_004603.1 (with corresponding nucleotide sequence at NM_004612.2) The protein sequence for the 426 amino acid isoform is published, for example, as GenBank accession number NP_001124388.1 (with corresponding nucleotide sequence at NM_001130916.1). Information regarding both isoforms is also published as UniProt entry P36897.

Human ALK1 is a 503 amino acid protein. The protein sequence is published, for example, as GenBank accession number NP_001070869.1 (with corresponding nucleotide sequence at NM_001077401.1; transcript variant 2) and NP_000011.2 (with corresponding nucleotide sequence at NM_000020.2; transcript variant 1), UniProt entry P37023.

Human ALK3 is a 532 amino acid protein. The protein sequence is published, for example, as GenBank accession number NP_004320 (with corresponding nucleotide sequence at NM_004329.2), UniProt entry P36894.

Human ALK4 has at least three isoforms. Isoform a is a 505 amino acid protein. The protein sequence is published, for example, as GenBank accession number NP_004293 (with corresponding nucleotide sequence at NM_004302), UniProt entry P36896.

Isoform a of human ALK6 is a 532 amino acid protein and isoform b is a 502 amino acid protein. The protein sequence for human ALK6 isoform a is published, for example, as GenBank accession number NP_001243722 (with corresponding nucleotide sequence at NM_001256793.1). The protein sequence for human ALK6 isoform b is published, for example, as GenBank accession number NP_001194 (with corresponding nucleotide sequence at NM_001203.2).

Note that each of the foregoing proteins are further processed in vivo, such as by the cleaving of a signal sequence, to yield a mature form.

### III. Pharmaceutical Compositions

Compounds of the present invention may be used in a pharmaceutical composition, e.g., combined with a pharmaceutically acceptable carrier, for administration to a patient. Such a composition may also contain diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. Such additional factors and/or agents may be included in the pharmaceutical composition to produce a synergistic effect with compounds of the invention, or to minimize side effects caused by the compound of the invention.

The pharmaceutical compositions of the invention may be in the form of a liposome or micelles in which compounds of the present invention are combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

The terms “pharmaceutically effective amount” or “therapeutically effective amount”, as used herein, means the total amount of each active component of the pharmaceutical composition or method that is sufficient to show a meaningful patient benefit, e.g., treatment, healing, prevention, inhibition or amelioration of a physiological response or condition, such as an inflammatory condition or pain, or an increase in rate of treatment, healing, prevention, inhibition or amelioration of such conditions. When applied to an individual active ingredient, administered alone, the term refers to that ingredient alone. When applied to a combination, the term refers to combined amounts of the active ingredients that result in the therapeutic effect, whether administered in combination, to serially or simultaneously.

Each of the methods of treatment or use of the present invention, as described herein, comprises administering to a mammal in need of such treatment or use a pharmaceutically or therapeutically effective amount of a compound of the present invention, or a pharmaceutically acceptable salt or ester form thereof. Compounds of the present invention may be administered in accordance with the method of the invention either alone or in combination with other therapies.

Administration of compounds of the present invention used in the pharmaceutical composition or to practice the method of the present invention can be carried out in a variety of conventional ways. Exemplary routes of administration that can be used include oral, parenteral, intravenous, intra-arterial, cutaneous, subcutaneous, intramuscular, topical, intracranial, intraorbital, ophthalmic, intravitreal, intraventricular, intracapsular, intraspinal, intracisternal, intraperitoneal, intranasal, aerosol, central nervous system (CNS) administration, or administration by suppository.

When a therapeutically effective amount of a compound(s) of the present invention is administered orally, compounds of the present invention may be in the form of a tablet, capsule, powder, solution or elixir. When administered in tablet form, the pharmaceutical composition of the invention may additionally contain a solid carrier such as a gelatin or an adjuvant. The tablet, capsule, and powder may contain from about 5 to 95% compound of the present invention, and preferably from about 10% to 90% compound of the present invention. When administered in liquid form, a liquid carrier such as water, petroleum, oils of animal or plant origin such as peanut oil, mineral oils, phospholipids, tweens, triglycerides, including medium chain triglycerides, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition typically contains from about 0.5 to 90% by weight of compound of the present invention, and preferably from about 1 to 50% compound of the present invention.

When a therapeutically effective amount of a compound(s) of the present invention is administered by intravenous, cutaneous or subcutaneous injection, compounds of the present invention may be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, cutaneous, or subcutaneous injection should contain, in addition to compounds of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of compound(s) of the present invention in the pharmaceutical composition of the present invention will depend upon the nature and severity of the condition being treated, and on the nature of prior treatments the patient has undergone. Ultimately, the practitioner will decide the amount of compound of the present invention with which to treat each individual patient. Initially, the practitioner may administer low doses of compound of the present invention and observe the patient's response. Larger doses of compounds of the present invention may be administered until the optimal therapeutic effect is obtained for the patient, and at that point the dosage is not increased further. It is contemplated that the various pharmaceutical compositions used to practice the method of the present invention should contain about 0.1 μg to about 100 mg (preferably about 0.1 mg to about 50 mg, more preferably about 1 mg to about 2 mg) of compound of the present invention per kg body weight.

The duration of intravenous therapy using the pharmaceutical composition of the present invention will vary, depending on the severity of the disease being treated and the condition and potential idiosyncratic response of each individual patient. It is contemplated that the duration of each application of the compounds of the present invention will be in the range of 12 to 24 hours of continuous intravenous administration. Ultimately the practitioner will decide on the appropriate duration of intravenous therapy using the pharmaceutical composition of the present invention.

IV. Use with Polymers

The compounds as disclosed herein may be conjugated to a polymer matrix, e.g., for controlled delivery of the compound. The compound may be conjugated via a covalent bond or non-covalent association. In certain embodiments wherein the compound is covalently linked to the polymer matrix, the linkage may comprise a moiety that is cleavable under biological conditions (e.g., ester, amide, carbonate, carbamate, imide, etc.). In certain embodiments, the conjugated compound may be a pharmaceutically acceptable salt, ester, or prodrug of a compound disclosed herein. A compound as disclosed herein may be associated with any type of polymer matrix known in the art for the delivery of therapeutic agents.

### V. Synthetic Preparation

The compounds disclosed herein can be prepared in a variety of ways known to one skilled in the art of organic synthesis, and in analogy with the exemplary compounds whose synthesis is described herein. The starting materials used in preparing these compounds may be commercially available or prepared by known methods. Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, which is incorporated herein by reference in its entirety.

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

### VI. Uses

BMPs and TGF-beta signaling pathways are essential to normal organogenesis and pattern formation, as well as the normal and pathological remodeling of mature tissues. Defects in the BMP signaling pathway are implicated in a number of congenital and acquired disease processes, including Hereditary Hemorrhagic Telangiectasia syndrome, Primary Pulmonary Hypertension or Pulmonary Arterial Hypertension, Juvenile Familial Polyposis, as well as sporadic renal cell and prostate carcinomas. It has been suggested that in certain disease states associated with defective signaling components, attenuated BMP signaling might be a cause, while our findings have suggested that in some contexts excess BMP signaling might be pathogenic (Waite et al. Nat. Rev. Genet. 4:763-773, 2005; Yu et. J. Biol. Chem. 280:24443-24450, 2003). The ability to modulate BMP signaling experimentally would provide a means for investigating therapy, and for determining the root causes of these conditions.

A. Treatment of Anemia. Including Iron Deficiency and Anemia of Chronic Disease

For a review, see Weiss et al. N. Engl. J. Med. 352:1011-1023, 2005. Anemia of inflammation (also called anemia of chronic disease) can be seen in patients with chronic infections, autoimmune diseases (such as systemic lupus erythematosis and rheumatoid arthritis, and Castleman's disease), inflammatory bowel disease, cancers (including multiple myeloma), and renal failure. Anemia of inflammation is often caused by maladaptive expression of the peptide hormone hepcidin. Hepcidin causes degradation of ferroportin, a critical protein that enables transport of iron from intracellular stores in macrophages and from intestinal epithelial cells. Many patients with renal failure have a combination of erythropoietin deficiency and excess hepcidin expression. BMP signaling induces expression of hepcidin and inhibiting hepcidin expression with BMP inhibitors increases iron levels. Compounds as described herein can be used to treat anemia due to chronic disease or inflammation and associated hyperhepcidinemic states.

The inflammatory cytokine IL-6 is thought to be the principal cause of elevated hepcidin expression in inflammatory states, based upon the elevation of IL-6 in anemia of inflammation of diverse etiologies, the effects of chronic IL-6 administration in vivo, and the protection against anemia in rodents deficient in IL-6 (Weiss et al. N. Engl. J. Med. 352:1011-1023, 2005). It has been shown that stimulating hepatoma cell lines with IL-6 induces hepcidin expression, while treatment with a BMP inhibitor abrogates IL-6-induced hepcidin expression (Yu et al. Nat. Chem. Biol. 4:33-41, 2008). Moreover, we have found that BMP inhibitors can inhibit hepcidin expression induced by injection of pathogenic bacteria in vivo. It has also been shown that systemic iron administration in mice and zebrafish rapidly activates BMP-responsive-SMADs and hepcidin expression in the liver, and that BMP antagonism effectively blocks these responses (Yu et al. Nat. Chem. Biol. 4:33-41, 2008). The functional importance of BMP signaling in iron regulation is supported by our finding that BMP inhibitors can inhibit hepcidin expression and raise serum iron levels in vivo. Taken together these data suggest that iron- and inflammation-mediated regulation of hepcidin and circulating iron levels require BMP signaling. Compounds as described herein may be used to alter iron availability in diverse circumstances for therapeutic benefit.

Compounds as described herein may be used in anemic states to (i) augment the efficacy of dietary iron or oral iron supplementation (which is safer than intravenous administration of iron) to increase serum iron concentrations; (ii) augment build-up of hemoglobin in the blood in anticipation of surgery or to enable blood donation for self in anticipation of surgery; (iii) enhance the efficacy of erythropoietin and its relatives, thereby enabling lower doses of erythropoietin to be administered for anemia while minimizing known toxicities and side effects of erythropoietin (i.e., hypertension, cardiovascular events, and tumor growth), and (iv) inhibit the hepcidin expression to help correct the anemia associated with inflammatory bowel disesease (Wang et al., Inflamm. Bowel Dis. 2012 January; 18(1):112-9. Epub 2011 Feb. 23).

B. Treatment of Fibrodysplasia Ossificans Progressiva (FOP)

FOP is caused by the presence of a constitutively-active mutant form of ALK2 in affected individuals (Shore et al. Nat. Genet. 38:525-527, 2006). A specific inhibitor of BMP signaling such as a compound as described herein can be used to prevent excessive bone formation in response to trauma, musculoskeletal stress or inflammation. Such a compound could also be used to aid in regression of pathologic bone. The BMP inhibitor could be administered systemically or locally to concentrate or limit effects to areas of trauma or inflammation.

A BMP inhibitor as described herein may be used as chronic therapy to suppress spontaneous bone formation in individuals who are highly susceptible. Transient therapy may be used to prevent abnormal bone formation in FOP individuals who develop osteomas or pathologic bone most frequently in association with trauma by administration before, during, or even after the traumatic incident. Transient therapy with BMP inhibitors as described herein could be used before, during or immediately after necessary or emergent medical or surgical procedures (and even important immunizations and tooth extractions) in individuals with FOP, to prevent pathologic calcification. Combination therapy with other bone inhibiting agents, immune modulatory or anti-inflammatory drugs (such as NSAIDs, steroids, cyclosporine, cyclophosphamide, azathioprine, methotrexate, rituxumab, etanercept, or similar drugs) may increase the effectiveness of BMP inhibitors in inhibiting heterotopic bone formation in this disorder.

A mouse model of FOP has been developed in which expression of a constitutively-active mutant form of ALK2 is induced by injecting the popliteal fossa of a genetically-modified mouse with an adenovirus directing expression of Cre recombinase. This model reproduces the ectopic calcification and disability seen in FOP patients.

C. Treatment of Cancers

Excessive BMP signaling, which could arise due to over-expression of BMPs, or, paradoxically, as a result of loss of BMP type II receptor expression, may contribute to the oncogenesis, growth or metastasis of certain solid tumors, including breast, prostate carcinomas, bone, lung, and renal cell carcinomas (Yu et al. J. Biol. Chem. 280:24443-24450, 2008; Waite et al. Nat. Rev. Genet. 4:763-773, 2003; Alarmo et al. Genes, Chromosomes Cancer 45:411-419, 2006; Kim et al. Cancer Res. 60:2840-2844, 2000; Kim et al. Clin. Cancer Res. 9:6046-6051, 2003; Kim et al. Oncogene 23:7651-7659, 2004). Inhibition of BMP9 signaling can prevent ovarian cancer cell growth (Herrera et al. Cancer Res. 2009 Dec. 15; 69(24):9254-62). Ovarian cancer growth is promoted by ALK2-SMAD signaling and could be inhibited by selective ALK2 inhibitors (Tsai et al. Cell Rep. 2012 Aug. 30; 2(2):283-93. Epub 2012 Aug. 9), such as with the compounds described herein. Diffuse intrinsic pontine gliomas (DIPG), non-brainstem high-grade gliomas, and other pediatric high-grade gliomas are frequently associated with aberrant signaling of the BMP pathway, e.g., through mutation of Alk-2. See, e.g., Wu, G. et al., Nat Genet. 2014 May; 46(5):444-50; Taylor, K. et al., Nat Genet. 2014 May; 46(5):457-61; Buczkowicz, P. et al., Nat Genet. 2014 May; 46(5):451-6; Fontebasso, A. M. et al., Nat Genet. 2014 May; 46(5):462-6; and Fangusaro, J., J Child Neurol. 2009 November; 24(11):1409-17. Accordingly, the compounds disclosed herein can be applied to the treatment of these cancers.

If increased BMP activity associated with BMP over-expression or BMP type II receptor deficiency contributes to the pathogenesis of disease, then inhibiting BMP signaling activity using compounds as described herein at the level of BMP type I receptors (downstream of both ligands and type II receptor) could be an effective means of normalizing BMP signaling activity and potentially inhibiting tumor growth or metastasis.

Compounds as described herein can be used to slow or arrest the growth or metastasis of such tumor cells (as well as other tumor constituent cell types) for clinical benefit, either as adjunctive or primary chemotherapy. Also, BMP inhibitors as described herein may be used to interfere with the bone metastatic properties of certain types of cancers (e.g., adenocarcinoma, such as prostate and breast carcinomas). In addition, compounds as described herein can be used to inhibit osteoblastic activity in tumors that either form bone or are bone-derived, such as osteosarcomas (as adjunctive or primary chemotherapy). Further, compounds as described herein can be used to inhibit osteoclastic activity (also regulated by BMPs through the action of its target gene RANKL), which is pathologically increased in conditions such as multiple myeloma and other bone-targeted tumors. Application of BMP inhibitors in these conditions may reduce the presence of osteolytic lesions and bone fractures due to tumor involvement.

D. Immune Modulation Via BMP Inhibitors

BMPs have been reported to attenuate the inflammatory or immune response (Choi et al. Nat. Immunol. 7:1057-1065, 2006; Kersten et al. BMC Immunol. 6:9, 2005), which can impair an individual's ability to fight infections (i.e., viral, bacterial, fungal, parasitic, or tuberculosis). Inhibitors of BMP signaling as described herein may thus augment the inflammatory or immune response enabling individuals to clear infections more rapidly.

Lymphocytes and other immune cells express BMP receptors on their cell surfaces, and there is growing evidence that BMPs regulate the development and maturation of various humoral and cellular immunologic compartments, and regulate humoral and cellular immune responses in mature organisms. The effects of BMP signals on immune cells are likely to be context-specific, as is commonly known for the effects of numerous cytokines of immunologic importance, and thus whether they augment or diminish the development or function of particular lymphocyte populations must be empirically determined. BMP antagonism using compounds as described herein may be an effective strategy for intentionally biasing the development of cellular, innate, or humoral immune compartments for therapy, or a strategy for the therapeutic deviation of immune responses in mature immune systems. These strategies may target inborn disorders of cellular, innate, or humoral immunity, or target disorders in which immune responses are inappropriately weak (e.g., as an adjuvant to promote successful antigen sensitization when immunization is difficult or ineffective by other means), or target disorders in which immune responses are excessive or inappropriate (e.g., autoimmunity and autosensitization). BMP inhibitors as described herein may also be effective in some contexts for the intentional induction of immune tolerance (i.e., in allotransplantation or autoimmunity) and for indications such as autoimmune diseases and inflammatory bowel disease (IBD) (Wang et al., Inflamm. Bowel to Dis. 2012 January; 18(1): 112-9. Epub 2011 Feb. 23). BMP inhibitors as described herein may also attenuate macrophage-mediated inflammation in response to Salmonella typhimurium in a model of inflammatory colitis (Wang L et al, J Clin Invest. 2009; 119(11):3322).

E. Treatment of Pathologic Bone Formation

Compounds as described herein can be used to ameliorate pathologic bone formation/bone fusion in inflammatory disorders, such as ankylosing spondylitis or other “seronegative” spondyloarthropathies, in which autoimmunity and inflammation in such disorders appear to stimulate bone formation. One application of the compounds would be to prevent excess bone formation after joint surgery, particularly in patients with ankylosing spondylitis or rheumatoid arthritis. Compounds as described herein can also be used to prevent calcinosis (dystrophic soft-tissue calcification) in diseases such as systemic lupus erythematosus, scleroderma, or dermatomyositis.

Blunt traumatic injury to muscles can cause abnormal bone formation within muscle in certain individuals, resulting in a disorder called myositis ossificans traumatica (Cushner et al. Orthop. Rev. 21:1319-1326, 1992.). Head trauma and burn injury can also induce heterotopic bone formation markedly impairing patient rehabilitation and recovery. Treatment with a BMP inhibitor as described herein, optionally in addition to anti-inflammatory medications usually prescribed for such a condition (e.g., non-steroidal anti-inflammatory drugs such as indomethacin or ibuprofen) may help to prevent the formation of pathologic bone in predisposed individuals, or to help lessen or regress lesions in individuals recently or remotely affected. Very rarely other muscles have been described to develop ossification in the presence of injury or trauma, including heart muscle, and similar treatment with a BMP inhibitor as described herein could be helpful in those circumstances.

F. Treatment of Ectopic or Maladaptive Bone Formation

BMP signals and their transcriptional targets are implicated in intimal and medial vascular remodeling and calcification in Monckeberg's vascular calcification disease and in atheromatous vascular disease (Bostrom et al. J. Clin. Invest. 91:1800-1809, 1993; Tyson et al. Arterioscler. Thromb. Vasc. Biol. 23:489-494, 2003). BMPs and BMP-induced osteodifferentation are also implicated in cardiac valvular calcification. Native cardiac valves can calcify particularly when they are already abnormal. A classic example is bicuspid aortic valve—these valves typically become calcified leading to stenosis. Patients with calcific aortic valve stenosis often require cardiac surgery for valve replacement. Abnormal calcification can adversely affect the function of prosthetic vascular grafts or cardiac valves. For example, prosthetic heart valves become calcified leading to narrowing and often leakage.

Compounds as described herein can be used to inhibit vascular or valvular calcific disease alone or in combination with atheromatous disease, renal disease, renal osteodystrophy or parathyroid disease.

Compounds as described herein can be used to inhibit calcification of prosthetic vascular or valvular materials by systemic or local administration or direct incorporation into prosthesis materials or other implants (e.g., in admixture with a polymer that coats or constitutes all or part of the implant or prosthesis).

In some instances, it is desired to delay fracture healing following a bone fracture, or to purposely inhibit fracture healing in certain locations to prevent impairment of function by maladaptive bone formation. For example, if a fracture occurs and for medical or practical reasons surgery cannot be performed immediately, fracture healing may be temporarily “suspended” by use of a BMP inhibitor as described herein, until definitive surgery or manipulation can be performed. This could prevent the need for subsequent intentional re-fracture in order to ensure correct apposition of bone fragments, for example. It is expected that upon stopping a BMP inhibitor normal fracture healing processes would ensue if the period of treatment is relatively short. In other cases, any amount of novel bone growth might impair function, such as when fracture affects a joint directly. In these cases, global or local inhibition of BMP activity (by systemic or local delivery of a BMP inhibitor as described herein via diffusion from a local implant or matrix) may be used to inhibit fracture healing or prevent fracture calluses at the critical areas.

G. Treatment of Skin Diseases

Expansion of cultured keratinocytes—In vitro, BMPs inhibit keratinocyte proliferation and promote differentiation (reviewed in Botchkarev et al. Differentiation 72:512-526, 2004). In patients in need of skin grafting (eg. after burns), skin grafts are made from cultured keratinocytes. The keratinocytes may be derived from other animals (xenografts), but these are only temporary as they will be rejected by the immune system. Keratinocytes can be derived from the patient themselves and can be grown into sheets of cells in the laboratory (cultured epithelial autografts). The patient will not reject keratinocytes derived from his/her own body. Addition of BMP inhibitors as described herein to keratinocyte cultures can be used to facilitate keratinocyte proliferation enabling patients to receive grafts sooner.

Improved epithelialization—BMP6 is highly expressed in skin injury, and high levels of BMP6 are detected in chronic human wounds of different etiologies (Kaiser et al. J. Invest. Dermatol. 111:1145-1152, 1998). In mice overexpressing BMP6 in their skin, reepithelialization and healing skin wounds were significantly delayed (Kaiser et at. J. Invest. Dermatol. 111:1145-1152, 1998). Improved epithelialization can reduce scar formation. Topical or systemic administration of BMP inhibitors as described herein can be used to augment epithelialization of skin wounds, for example, in the treatment of pressure ulcers (bed sores) or non-healing or poorly-healing skin ulcers (e.g., in patients with peripheral vascular disease, diabetes mellitus, venous incompetence). Compounds would also be expected to decrease scar formation.

Promotion of hair growth—Growth of hair follicles on the scalp is cyclic with three phases: anagen (the growth phase), catagen (the involutional phase), and telogen (resting phase). Recent evidence suggests that BMP signals delay the transition from telogen to anagen (Plikus et al. Nature 451:340-344, 2008). Inhibition of BMP signaling using compounds as described herein can shorten the telogen phase and increase the number of follicles in the anagen phase. Compounds as described herein can be used to treat circumstances wherein hair follicles are insufficient or when hairs are being lost more frequently than they are grown. These circumstances include androgenetic alopecia (male pattern balding), alopecia areata, and telogen effluvium.

Treatment of psoriasis—Psoriasis is an inflammatory skin disorder which sometimes occurs following skin trauma and the ensuing repair and inflammation (Koebner phenomenon). BMPs may participate in repair and inflammatory mechanisms that cause psoriasis, since over-expression of BMP6 in the skin of mice leads to skin lesions similar to those seen in patients with psoriasis (Blessing et al. J. Cell. Biol. 135:227-239, 1996). Compounds as described herein may be administered topically or systemically to treat established psoriasis or prevent its development after skin injury.

Treatment of corneal scarring—BMP6 expression is associated with conjunctival scarring (Andreev et al. Exp. Eye Res. 83:1162-1170, 2006). Compounds as described herein can be used to prevent or treat corneal scarring and the resulting blindness.

H. Treatment of Systemic Hypertension

Infusion of BMP4 induces systemic hypertension in mice (Miriyala et al. Circulation 113:2818-2825, 2006). Vascular smooth muscle cells express a variety of BMP ligands. BMPs increase the expression of voltage gated potassium channels and thereby increase constriction of vascular smooth muscle (Fantozzi et al. Am. J. Physiol. Lung Cell. Mol. Physiol. 291:L993-1004, 2006). Compounds as described herein that inhibit BMP signaling can be used to reduce blood pressure. Sustained reduction of blood pressure in patients with hypertension would be expected to prevent myocardial infarction, congestive heart failure, cerebrovascular accidents, and renal failure. BMP inhibitors as described herein can be used to target the hypertension in specific vascular beds, such as in pulmonary hypertension via local delivery (e.g., via aerosol).

I. Treatment of Pulmonary Hypertension

BMP signaling contributes to the pathogenesis of pulmonary hypertension. For example, mice with decreased BMP4 levels are protected from the pulmonary hypertension and pulmonary vascular remodeling induced by breathing low oxygen concentrations for prolonged periods (Frank et al. Circ. Res. 97:496-504, 2005). Moreover, mutations in the gene encoding the type II BMP receptor (BMPRII) are frequently found in patients with sporadic and familial pulmonary arterial hypertension. It might be anticipated that decreased BMP signaling might cause pulmonary hypertension. However, Yu and colleagues (Yu et al. J. Biol. Chem. 280:24443-24450, 2008) reported that BMPRII deficiency paradoxically increases BMP signaling by subsets of BMP ligands, and thus increased BMP signaling using compounds as described herein may actually contribute to the development of pulmonary hypertension.

Compounds as described herein can be used to prevent the development of pulmonary arterial hypertension in patients at risk for the disease (e.g., patients with BMPRII mutations) or to treat patients with idiopathic or acquired pulmonary arterial hypertension. Decreased pulmonary hypertension in individuals treated with the compounds described herein would be expected to decrease shortness of breath, right ventricular hypertrophy, and right ventricular failure.

J. Treatment of Ventricular Hypertrophy

BMP-10 levels are increased in the hypertrophied ventricles of rats with hypertension, and this BMP ligand induces hypertrophy in cultured neonatal rat ventricular myocytes (Nakano et al. Am. J. Physiol. Heart. Circ. Physiol. 293:H3396-3403, 2007). Sun et al. (Hypertension 2013 February; 61(2):352-60) suggest that small molecule BMP inhibitors can reduce adverse left ventricular remodeling (hypertrophy). Inhibition of BMP-10 signaling with compounds as described herein can to prevent/treat ventricular hypertrophy. Ventricular hypertrophy can lead to congestive heart failure due to diastolic dysfunction. Compounds described herein would be expected to prevent/treat congestive heart failure.

K. Treatment of Neurologic Disorders

Treatment of spinal cord injury and neuropathy—BMPs are potent inhibitors of axonal regeneration in the adult spinal cord after spinal cord injury (Matsuura et al. J. Neurochem. 2008). Expression of BMPs is reported to be elevated in oligodendrocytes and astrocytes around the injury site following spinal cord contusion. Intrathecal administration of noggin, a BMP inhibitor, led to enhanced locomotor activity and significant regrowth of the corticospinal tract after spinal cord contusion.

RGMa inhibits axonal growth and recovery after spinal cord injury, as well as synapse re-formation, effects which are blocked by an antibody directed against RGMa (Hata et al. J. Cell. Biol. 173:47-58, 2006; Kyoto et al. Brain Res. 1186:74-86, 2007). RGMa enhances BMP signaling (Babitt et al. J. Biol. Chem. 280:29820-29827, 2005) suggesting that BMP signaling may be responsible for preventing axonal growth and recovery.

Based on these considerations, compounds as described herein would be expected to increase axonal growth and recovery after spinal cord injury. Compounds as described herein would be expected to prevent/treat neuropathies associated with a wide spectrum of disorders including diabetes mellitus. Compounds as described herein would be expected to treat both the pain and motor dysfunction associated with neuropathies.

Treatment of neurologic disorders associated with central nervous system inflammation—BMP4 and 5 have been detected in multiple sclerosis and Creutzfeldt-Jakob disease lesions (Deininger et al. Acta Neuropathol. 90:76-79, 1995). BMPs have also been detected in mice with experimental autoimmune encephalomyelitis, an animal model of multiple sclerosis (Ara et al. J. Neurosci. Res. 86:125-135, 2008). Compounds as described herein may be used to prevent or treat multiple sclerosis as well as other neurologic disorders associated with central nervous system inflammation, or maladaptive injury repair processes mediated by BMP signals.

Treatment of dementias—Inhibitors of BMP signaling can promote neurogenesis in mouse neural precursor cells (Koike et al. J. Biol. Chem. 282:15843-15850, 2007). Compounds as described herein can be used to augment neurogenesis in a variety of neurologic disorders associated with accelerated loss of neurons including cerebrovascular accidents and Alzheimer's Disease, as well as other dementias.

Altering memory and learning—BMP signaling has an important role in the development and maintenance of neurons involved in memory and cognitive behavior. For example, mice deficient in the BMP inhibitor, chordin, have enhanced spatial learning but less exploratory activity in a novel environment (Sun et al. J. Neurosci. 27:7740-7750, 2007). Compounds as described herein can be used to alter or prevent memory or learning, for example, inducing amnesia for anesthesia or in other situations likely to cause distress, or to prevent Post-Traumatic Stress Disorder.

L. Treatment of Atherosclerosis

Abundant evidence suggests that BMP ligands are pro-inflammatory and pro-atherogenic in the blood vessel wall (Chang et al. Circulation 116:1258-1266, 2007). Knocking-down expression of BMP4 decreased inflammatory signals, whereas knocking-down BMP inhibitors (e.g., follistatin or noggin) increased inflammatory signals. Compounds as described herein can be used to reduce vascular inflammation associated with atherosclerosis, autoimmune disease, and other vasculitides. By decreasing atherosclerosis, it would be anticipated that compounds as described herein would decrease the incidence and/or severity of acute coronary syndromes (angina pectoris and heart attack), transient ischemic attacks, stroke, peripheral vascular disease, and other vascular ischemic events. Moreover, in so far as atherosclerosis contributes to the pathogenesis of aneurysm formation, compounds as described herein can be used to slow the progression of aneurysm formation decreasing the frequency of aneurismal rupture and the requirement for surgery.

As BMPs and many of the BMP-induced gene products that affect matrix remodeling are overexpressed in early atherosclerotic lesions, BMP signals may promote atherosclerotic plaque formation and progression (Bostrom et al. J Clin Invest. 91: 1800-1809. 1993: Dhore et al. Arterioscler Thromb Vasc Biol. 21: 1998-2003. 2001). BMP signaling activity in the atheromatous plaque may thus represent a form of maladaptive injury-repair, or may contribute to inflammation. Over time, BMP signals may also induce resident or nascent vascular cell populations to differentiate into osteoblast-like cells, leading to intimal and medial calcification of vessels (Hruska et al. Circ Res. 97: 105-112. 2005). Calcific vascular disease, or arteriosclerosis, is associated with decreased vascular distensibility, and increased risk of cardiovascular events and mortality, and is particularly problematic when associated with underlying atherosclerotic disease (Bostrom et al. Crit Rev Eukaryot Gene Expr. 10: 151-158. 2000). Both atherosclerotic and calcific lesions may be amenable to regression, however, if signals which contribute to their progression can be intercepted (Sano et al. Circulation. 103: 2955-2960. 2001). In certain aspects, inhibitor of BMP type I receptor activity may be used to limit the progression of atheromatous plaques and vascular calcification in vivo (Derwall et al. Arteriosclerosis, Thrombosis, and Vascular Biology. 2012; 32: 613-622).

M. Treatment of Hypercholesterolemia or Hyperlipoproteinemia

Treatment with small molecule or recombinant BMP inhibitors reduces vascular inflammation (via macrophage accumulation and cathepsin activity), atheroma formation, and vascular calcification in mice deficient in low-density lipoprotein receptor (LDLR^{-/-}). Without wishing to be bound by theory, as potential explanations for impact on vascular inflammation, oxidized LDL (oxLDL) has been found to increase BMP2 expression and induce the production of reactive oxygen species (ROS) in human aortic endothelial cells. ROS production induced by oxLDL appears to require BMP signaling, based on inhibition by small molecule or recombinant BMP inhibitors. Treatment with small molecule BMP inhibitors reduces plasma low-density lipoprotein levels without inhibiting HMG-CoA reductase activity, suggesting a role of BMP signaling in the regulation of LDL cholesterol biosynthesis. Small molecule BMP inhibitors have also been found to inhibit hepatosteatosis seen in LDLR-deficient mice fed a high-fat diet. Small molecule or recombinant BMP inhibitors inhibit the synthesis of ApoB-100 in hepatoma cells in vitro. These findings implicate BMP signaling in vascular calcification and atherogenesis and provide at least two novel mechanisms by which BMP signaling may contribute to the pathogenesis of atherosclerosis. These studies highlight the BMP signaling pathway as a therapeutic target in the treatment of atherosclerosis while identifying several novel functions of BMP signaling in the regulation of vascular oxidative stress, inflammation and lipid metabolism.

In certain embodiments, BMP inhibitors as described herein may be used for the reduction of circulating levels of ApoB-100 in patients. In certain embodiments, BMP inhibitors as described herein may be used for the reduction of circulating levels of LDL in patients. Accordingly, BMP inhibitors as described herein may be used for the treatment of hypercholesterolemia, hyperlipidemia, or hyperlipoproteinemia, including congenital or acquired hypercholesterolemia, hyperlipidemia, or hyperlipoproteinemia.

In certain embodiments, the congenital hypercholesterolemia, hyperlipidemia, or hyperlipoproteinemia is autosomal dominant hypercholesterolemia (ADH), familial hypercholesterolemia (FH), polygenic hypercholesterolemia, familial combined hyperlipidemia (FCHL), hyperapobetalipoproteinemia, or small dense LDL syndrome (LDL phenotype B).

In certain embodiments, the acquired hypercholesterolemia, hyperlipidemia, or hyperlipoproteinemia is associated with diabetes mellitus, hyperlipidemic diet and/or sedentary lifestyle, obesity, metabolic syndrome, intrinsic or secondary liver disease, primary biliary cirrhosis or other bile stasis disorders, alcoholism, pancreatitis, nephrotic syndrome, endstage renal disease, hypothyroidism, iatrogenesis due to administration of thiazides, beta-blockers, retinoids, highly active antiretroviral agents, estrogen, progestins, or glucocorticoids. In certain embodiments, BMP inhibitors as described herein may be used for the treatment of diseases, disorders, or syndromes associated with defects in lipid absorption or metabolism, such as sitosterolemia, cerebrotendinous xanthomatosis, or familial hypobetalipoproteinemia.

In certain embodiments, BMP inhibitors as described herein may be used for the treatment of diseases, disorders, or syndromes caused by hyperlipidemia, such as coronary artery disease and its manifestations (e.g., myocardial infarction; angina pectoris; acute coronary artery syndromes, such as unstable angina pectoris; cardiac dysfunction, such as congestive heart failure, caused by myocardial infarction; or cardiac arrhythmia associated with myocardial ischemia/infarction), stroke due to occlusion of arteries supplying portions of the brain, cerebral hemorrhage, peripheral arterial disease (e.g., mesenteric ischemia; renal artery stenosis; limb ischemia and claudication; subclavian steal syndrome; abdominal aortic aneurysm; thoracic aortic aneurysm, pseudoaneurysm, intramural hematoma; or penetrating aortic ulcer, aortic dissection, aortic stenosis, vascular calcification, xanthoma, such as xanthoma affecting tendons or scleral and cutaneous xanthomas, xanthelasma, or hepatosteatosis.

In certain embodiments, BMP inhibitors as described herein may be used for the treatment of the foregoing diseases, disorders, or syndromes regardless of circulating lipid levels, such as in individuals exhibiting normal circulating lipid levels or metabolism.

In certain embodiments, BMP inhibitors as described herein may be used for the reduction of secondary cardiovascular events arising from coronary, cerebral, or peripheral vascular disease. In certain such embodiments, BMP inhibitors as described herein may be used to treat individuals regardless of lipid levels, such as used in the treatment of individuals exhibiting normal circulating cholesterol and lipid levels. In certain such embodiments, BMP inhibitors as described herein are administered conjointly with a HMG-CoA reductase inhibitor.

In certain embodiments, BMP inhibitors as described herein may be used for the prevention of cardiovascular disease, such as in individuals with elevated markers of cardiovascular risk (e.g., C-reactive protein) or, for example, an elevated Framingham Risk Score. In certain such embodiments, BMP inhibitors as described herein may be used to prevent cardiovascular disease in individuals exhibiting normal circulating cholesterol and lipid levels.

In certain embodiments wherein one or more BMP inhibitors as described herein are used in the treatment or prevention of the foregoing diseases, disorders, or syndromes, the patient being treated is not diagnosed with and/or is not suffering from one or more of the following conditions: vascular inflammation associated with atherosclerosis, automimmune disease, and other vasculitides; atherosclerotic disease, atheromatous plaques, and/or vascular calcification; an aneurysm and/or aneurysm formation; acute coronary syndromes (angina pectoris and heart attack), transient ischemic attacks, stroke, peripheral vascular disease, or other vascular ischemic events.

In other embodiments wherein one or more BMP inhibitors as described herein are used in the treatment or prevention of the foregoing diseases, disorders, or syndromes (e.g., for the reduction of circulating levels of ApoB-100 and/or LDL in patients; for the treatment of hypercholesterolemia, hyperlipidemia, or hyperlipoproteinemia, including congenital or acquired hypercholesterolemia, hyperlipidemia, or hyperlipoproteinemia; for the treatment of diseases, disorders, or syndromes associated with defects in lipid absorption or metabolism; for the treatment of diseases, disorders, or syndromes caused by hyperlipidemia; for the reduction of secondary cardiovascular events arising from coronary, cerebral, or peripheral vascular disease; or for the reduction of secondary cardiovascular events arising from coronary, cerebral, or peripheral vascular disease), the patient being treated is also diagnosed with and/or is also suffering from one or more of the following conditions: vascular inflammation associated with atherosclerosis, automimmune disease, and other vasculitides; atherosclerotic disease, atheromatous plaques, and/or vascular calcification; an aneurysm and/or aneurysm formation; acute coronary syndromes (angina pectoris and heart attack), transient ischemic attacks, stroke, peripheral vascular disease, or other vascular ischemic events.

N. Propagation, Engraftment and Differentiation of Progenitor Cells Including Embryonic and Adult Stem Cells In Vitro and In Vivo

BMP signals are crucial for regulating the differentiation and regeneration of precursor and stem cell populations, in some contexts and tissues preventing (while in other contexts directing) differentiation towards a lineage. Compounds as described herein can be used to (i) maintain a pluripotential state in stem cell or multipotent cell populations in vivo or in vitro; (ii) expand stem cell or multipotent cell populations in vivo or in vitro; (iii) direct differentiation of stem cell or multipotent cell populations in vivo or in vitro; (iv) manipulate or direct the differentiation of stem cell or multipotent cell populations in vivo or in vitro, either alone or in combination or in sequence with other treatments; and (v) modulate the de-differentiation of differentiated cell populations into multipotent or progenitor populations.

Numerous stem cell and precursor lineages require BMP signals in order to determine whether they will expand, differentiate towards specific tissue lineages, home in and integrate with particular tissue types, or undergo programmed cell death. Frequently BMP signals interact with signals provided by growth factors (bFGF, PDGF, VEGF, HBEGF, PIGF, and others), Sonic Hedgehog (SHH), notch, and Wnt signaling pathways to effect these changes (Okita et al. Curr. Stem Cell Res. Ther. 1:103-111, 2006). Compounds as described herein can be used to direct the differentiation of stem cells (e.g., embryonic stem cells) or tissue progenitor cells towards specific lineages for therapeutic application (Park et al. Development 131:2749-2762, 2004; Pashmforoush et al. Cell 117:373-386, 2004). Alternatively for certain cell populations, BMP inhibitors as described herein may be effective in preventing differentiation and promoting expansion, in order to produce to sufficient numbers of cells to be effective for a clinical application. The exact combination of BMP inhibitor and growth factor or signaling molecule may be highly specific to each cell and tissue type.

For example, certain embryonic stem cell lines require co-culture with leukemia inhibitory factor (LIF) to inhibit differentiation and maintain the pluripotency of certain cultured embryonic stem cell lines (Okita et al. Curr. Stem Cell Res. Ther. 1:103-111, 2006). Use of a BMP inhibitor as described herein may be used to maintain pluripotency in the absence of LIF. Other ES cell lines require coculture with a specific feeder cell layer in order to maintain pluripotency. Use of a BMP inhibitor as described herein, alone or in combination with other agents, may be effective in maintaining pluripotency when concerns of contamination with a feeder cell layer, or its DNA or protein components would complicate or prevent use of cells for human therapy.

In another example, in some circumstances antagonizing BMP signals with a protein such as noggin shortly before cessation of LIF in culture is able to induce differentiation into a cardiomyocyte lineage (Yuasa et al. Nat. Biotechnol. 23:607-611, 2005). Use of a pharmacologic BMP inhibitor as described herein may achieve similar if not more potent effects. Such differentiated cells could be introduced into diseased myocardium therapeutically. Alternatively, such treatment may actually be more effective on engrafted precursor cells which have already homed in to diseased myocardium. Systemic therapy with a protein inhibitor of BMP such as noggin would be prohibitively expensive and entail complicated dosing. Delivery of a BMP inhibitor as described herein, systemically or locally, could bias the differentiation of such precursor cells into functioning cardiomyocytes in situ.

O. Treatment of Cartilage Defects

The selective inhibition of specific BMP receptors enables cartilage formation by preventing calcification and mineralization of scaffolds produced by mesenchymal stem cells (Hellingman et al. Tissue Eng Part A. 2011 April; 17(7-8):1157-67. Epub 2011 Jan. 17.) Accordingly, compounds of the invention may be useful to promote cartilage repair/regeneration in patients with cartilage injuries or defects, as well as in the ex vivo or in vitro production of cartilage tissue, e.g., for implantation, from appropriate cells, such as mesenchymal stem cells.

P. Application of Compounds with Varying Degrees of Selectivity: Compounds which Inhibit BMP Signaling Via Particular BMP Type 1 Receptors, or Compounds which Also Affect Signaling Via TGF-β, Activin, AMP Kinase, or VEGF Receptors

ALK-specific inhibitors—Dorsomorphin inhibits the activity of the BMP type I receptors, ALK2, ALK3, and ALK6. Dorsomorphin inhibits ALK2 and ALK3 to a greater extent than it does ALK6 (Yu et al. Nat. Chem. Biol. 4:33-41, 2008). Several of the compounds described herein will have relative greater selectivity for particular BMP type I receptors. The pathogenesis of certain diseases might be attributed to the dysfunctional signaling of one particular receptor. For example, fibrodysplasia ossificans progressiva is a disease caused by aberrant (constitutively active) ALK2 function (Yu et al. Nat. Chem. Biol. 4:33-41, 2008). In such instances, compounds as described herein which specifically antagonize the function of a subset of the BMP type I receptors may have the advantage of reduced toxicity or side effects, or greater effectiveness, or both.

Some compounds as described herein may have a high degree of selectivity for BMP vs. TGF-β, Activin, AMP kinase, and VEGF receptor signaling. Other compounds may be less specific and may target other pathways in addition to BMP signaling. In the treatment of tumors, for example, agents which inhibit BMP signaling as well as one or more of the above pathways can have beneficial effects (e.g., decrease tumor size), when molecular phenotyping of specific patients' tumors reveals dysregulation of multiple pathways.

Some compounds as described herein have a high degree of selectivity for ALK2 versus ALK1 or ALK3 or ALK4 or ALK5 or ALK6. Selective inhibition of ALK2 versus ALK1 or ALK3 or ALK4 or ALK5 or ALK6 may minimize unwanted effects or toxicity. Chronic ALK3 inhibition might impair normal mucosal epithelial turnover due to known importance in intestinal crypt stem cell recycling, and implication of ALK3 function in juvenile familial polyposis. ALK1 inhibition might impair normal vascular remodeling and lead to complications similar to human hereditary telangiectasia syndrome type 2 (HHT2), such as leaky capillaries, AV malformations, and bleeding. Accordingly, compounds that selectively inhibit ALK2 relative to ALK3 and ALK1 may help avoid toxicities of this type that might be encountered through the use of an unselective inhibitor.

In certain embodiments, the invention provides a method of inhibiting the activity of ALK2 in a human, comprising administering to the human a small molecule that selectively inhibits the activity of human ALK2 relative to the activity of human ALK1. In some such embodiments, the small molecule inhibits the activity of human ALK2 with an IC₅₀ that is lower by a factor of about 2 than its IC₅₀ for inhibiting the activity of human ALK1. In some such embodiments, the small molecule inhibits the activity of human ALK2 with an IC₅₀ that is lower by a factor of 5 than its IC₅₀ for inhibiting the activity of human ALK1. In some such embodiments, the small molecule inhibits the activity of human ALK2 with an IC₅₀ that is lower by a factor of 10 than its IC₅₀ for inhibiting the activity of human ALK1. In some such embodiments, the small molecule inhibits the activity of human ALK2 with an IC₅₀ that is lower by a factor of 15 or 20 or 30 or 40 or 50 or 100 or 200 or 300 or 400 or 500 or 600 or 800 or 1000 or 1500 or 2000 or 5000 or 10000 or 15,000 or 20,000 or 40,000 or 50,000 or 60,000 or 70,000 or 80,000 or 90,000 or 100,000 than its IC₅₀ for inhibiting the activity of human ALK1.

In certain embodiments, the small molecule has a structure of Formula I or II as described herein.

In certain embodiments, the invention provides a method of inhibiting the activity of ALK2 in a human, comprising administering to the human a small molecule that selectively inhibits the activity of human ALK2 relative to the activity of human ALK3. In some such embodiments, the small molecule inhibits the activity of human ALK2 with an IC₅₀ that is lower by a factor of 15 than its IC₅₀ for inhibiting the activity of human ALK3. In some such embodiments, the small molecule inhibits the activity of human ALK2 with an IC₅₀ that is lower by a factor of 20 than its IC₅₀ for inhibiting the activity of human ALK3. In some such embodiments, the small molecule inhibits the activity of human ALK2 with an IC₅₀ that is lower by a factor of 30 than its IC₅₀ for inhibiting the activity of human ALK3. In some such embodiments, the small molecule inhibits the activity of human ALK2 with an IC₅₀ that is lower by a factor of 50 or 100 or 200 or 300 or 400 or 500 or 600 or 800 or 1000 or 1500 or 2000 or 5000 or 10000 or 15,000 or 20,000 or 40,000 or 60,000 or 70,000 or 80,000 or 90,000 or 100,000 than its IC₅₀ for inhibiting the activity of human ALK3.

In certain embodiments, the small molecule has a structure of Formula I or II as described herein.

In certain embodiments, the invention provides a method of inhibiting the activity of ALK2 in a human, comprising administering to the human a small molecule that selectively inhibits the activity of human ALK2 relative to the activity of human ALK4. In some such embodiments, the small molecule inhibits the activity of human ALK2 with an IC₅₀ that is lower by a factor of 1000 than its IC₅₀ for inhibiting the activity of human ALK4. In some such embodiments, the small molecule inhibits the activity of human ALK2 with an IC₅₀ that is lower by a factor of 2000 than its IC₅₀ for inhibiting the activity of human ALK4. In some such embodiments, the small molecule inhibits the activity of human ALK2 with an IC₅₀ that is lower by a factor of 3000 than its IC₅₀ for inhibiting the activity of human ALK4. In some such embodiments, the small molecule inhibits the activity of human ALK2 with an IC₅₀ that is lower by a factor of 4000 or 5000 or 6000 or 7000 or 8000 or 9000 or 10,000 or 12,000 or 14,000 or 16,000 or 18,000 or 20,000 or 25,000 or 30,000 or 40,000 or 50,000 or 60,000 or 70,000 or 80,000 or 90,000 or 100,000 than its IC₅₀ for inhibiting the activity of human ALK4.

In certain embodiments, the small molecule has a structure of Formula I or II as described herein.

In certain embodiments, the invention provides a method of inhibiting the activity of ALK2 in a human, comprising administering to the human a small molecule that selectively inhibits the activity of human ALK2 relative to the activity of human ALK6. In some such embodiments, the small molecule inhibits the activity of human ALK2 with an IC₅₀ that is lower by a factor of 2 than its IC₅₀ for inhibiting the activity of human ALK6. In some such embodiments, the small molecule inhibits the activity of human ALK2 with an IC₅₀ that is lower by a factor of 5 than its IC₅₀ for inhibiting the activity of human ALK6. In some such embodiments, the small molecule inhibits the activity of human ALK2 with an IC₅₀ that is lower by a factor of 10 than its IC₅₀ for inhibiting the activity of human ALK6. In some such embodiments, the small molecule inhibits the activity of human ALK2 with an IC₅₀ that is lower by a factor of 15 or 20 or 30 or 40 or 50 or 100 or 200 or 300 or 400 or 500 or 600 or 800 or 1000 or 1500 or 2000 or 5000 or 10000 or 15,000 or 20,000 or 40,000 or 50,000 or 60,000 or 70,000 or 80,000 or 90,000 or 100,000 than its IC₅₀ for inhibiting the activity of human ALK6.

In certain embodiments, the small molecule has a structure of Formula I or II as described herein.

In one aspect, the invention provides a method of inhibiting the activity of ALK2 in a human, comprising administering to the human a small molecule that selectively inhibits the activity of human ALK2 relative to the activity of human ALK5. In some such embodiments, the small molecule inhibits the activity of human ALK2 with an IC₅₀ that is lower by a factor of 1000 than its IC₅₀ for inhibiting the activity of human ALK5. In some such embodiments, the small molecule inhibits the activity of human ALK2 with an IC₅₀ that is lower by a factor of 2000 than its IC₅₀ for inhibiting the activity of human ALK5. In some such embodiments, the small molecule inhibits the activity of human ALK2 with an IC₅₀ that is lower by a factor of 3000 than its IC₅₀ for inhibiting the activity of human ALK5. In some such embodiments, the small molecule inhibits the activity of human ALK2 with an IC₅₀ that is lower by a factor of 4000 or 5000 or 6000 or 7000 or 8000 or 9000 or 10,000 or 12,000 or 14,000 or 16,000 or 18,000 or 20,000 or 25,000 or 30,000 or 40,000 or 50,000 or 60,000 or 70,000 or 80,000 or 90,000 or 100,000 than its IC₅₀ for inhibiting the activity of human ALK5.

In certain embodiments, the small molecule has a structure of Formula I or II as described herein.

Compounds as described herein can be used to treat subjects (e.g., humans, domestic pets, livestock, or other animals) by use of dosages and administration regimens that are determined to be appropriate by those of skill in the art, and these parameters may vary depending on, for example, the type and extent of the disorder treated, the overall health status of the subject, the therapeutic index of the compound, and the route of administration. Standard clinical trials can be used to optimize the dose and dosing frequency for any particular pharmaceutical composition of the invention. Exemplary routes of administration that can be used include oral, parenteral, intravenous, intra-arterial, subcutaneous, intramuscular, topical, intracranial, intraorbital, ophthalmic, intraventricular, intracapsular, intraspinal, intracisternal, intraperitoneal, intranasal, aerosol, or administration by suppository. Methods for making formulations that can be used in the invention are well known in the art and can be found, for example, in Remington: The Science and Practice of Pharmacy (20th edition, Ed., A. R. Gennaro), Lippincott Williams & Wilkins, 2000.

Q. Combination Therapies

In certain instances BMP inhibitors as described herein may be used in combination with other current or future drug therapies, because the effects of inhibiting BMP alone may be less optimal by itself, and/or may be synergistic or more highly effective in combination with therapies acting on distinct pathways which interact functionally with BMP signaling, or on the BMP pathway itself. In certain instances, conjoint administration of a BMP inhibitor as described herein with an additional drug therapy reduces the dose of the additional drug therapy such that it is less than the amount that achieves a therapeutic effect when used in a monotherapy (e.g., in the absence of a BMP inhibitor as described herein). Some examples of combination therapies could include the following.

In certain embodiments, BMP inhibitors as described herein may be administered conjointly with other antihyperlipidemic agents or antilipidemic agents including, but not limited to, HMG-CoA reductase inhibitors (e.g., atorvastatin, cerivastatin, fluvastatin, lovastatin, mevastatin, pitavastain, pravastatin, rosuvastatin, or simvastatin), fibrates (e.g., bezafibrate, ciprofibrate, clofibrate, gemfibrozil, or fenofibrate), ezetimibe, niacin, cholesteryl ester transfer protein (CETP) inhibitors (e.g., torcetrapib, anacetrapib, or dalcetrapib), cholestyramine, colestipol, probucol, dextrothyroxine, bile acid sequestrants, or combinations of the above.

In certain embodiments, BMP inhibitors as described herein may be administered conjointly with a treatment for diabetes including, but not limited to, sulfonyl ureas (e.g., chlorpropamide, tolbutamide, glyburide, glipizide, or glimepiride), medications that decrease the amount of glucose produced by the liver (e.g., metformin), meglitinides (e.g., repaglinide or nateglinide), medications that decrease the absorption of carbohydrates from the intestine (e.g., alpha glucosidase inhibitors such as acarbose), medications that effect glycemic control (e.g., pramlintide or exenatide), DPP-IV inhibitors (e.g., sitagliptin), insulin treatment, thiazolidinones (e.g., troglitazone, ciglitazone, pioglitazone, or rosiglitazone), oxadiazolidinediones, alpha-glucosidase inhibitors (e.g., miglitol or acarbose), agents acting on the ATP-dependent potassium channel of the beta cells (e.g., tolbutamide, glibenclamide, glipizide, glicazide, or repaglinide), nateglinide, glucagon inhibitors, inhibitors of hepatic enzymes involved in stimulation of gluconeogenesis and/or glycogenolysis, or combinations of the above.

In certain embodiments, BMP inhibitors as described herein may be administered conjointly with a treatment for obesity including, but not limited to, orlistat, sibutramine, phendimetrazine, phentermine, diethylpropion, benzphetamine, mazindol, dextroamphetamine, rimonabant, cetilistat, GT 389-255, APD356, pramlintide/AC 137, PYY3-36, AC 162352/PYY3-36, oxyntomodulin, TM 30338, AOD 9604, oleoyl-estrone, bromocriptine, ephedrine, leptin, pseudoephedrine, or pharmaceutically acceptable salts thereof, or combinations of the above.

In certain embodiments, BMP inhibitors as described herein may be administered conjointly with an antihypertensive agent including, but not limited to, beta-blockers (e.g., alprenolol, atenolol, timolol, pindolol propranolol and metoprolol), ACE (angiotensin converting enzyme) inhibitors (e.g., benazepril, captopril, enalapril, fosinopril, lisinopril, quinapril and ramipril), calcium channel blockers (e.g., nifedipine, felodipine, nicardipine, isradipine, nimodipine, diltiazem and verapamil), and alpha-blockers (e.g., doxazosin, urapidil, prazosin and terazosin), or combinations of the above.

In certain embodiments, BMP inhibitors as described herein may be administered conjointly with a treatment for anemia (e.g., anemia of inflammation associated with renal failure and hemodialysis), including but not limited to erythopoiesis-stimulating agents (e.g. erythropoietin).

Tyrosine kinase receptor inhibitors, such as SU-5416, and BMP inhibitors as described herein may have synergistic effects at inhibiting angiogenesis, particularly for anti-angiogenic therapy against tumors. BMP signals (BMP-4) are thought to be critical for the commitment of stem or precursor cells to a hematopoietic/endothelial common progenitor, and may promote the proliferation, survival, and migration of mature endothelial cells necessary for angiogenesis (Park et al. Development 131:2749-2762, 2004). Thus antagonism of BMP signals using compounds as described herein may provide additional inhibition of angiogenesis at the level of endothelial precursors and cells. Similarly, co-treatment with BMP inhibitors as described herein and other tyrosine kinase receptor inhibitors such as imatinib (Gleevec) could be used to inhibit vascular remodeling and angiogenesis of certain tumors.

The combination of a sonic hedgehog agonist and a BMP inhibitor as described herein may be particularly useful for promoting hair growth, as SHH activity is known to stimulate the transition of follicles out of telogen (resting) phase (Paladini et al. J. Invest. Dermatol. 125:638-646, 2005), while inhibiting the BMP pathway shortens the telogen phase (Plikus et at. Nature 451:340-344, 2008). The use of both would be expected to cause relatively increased time in the anagen or growth phase.

Combined use of Notch modulators (e.g., gamma-secretase inhibitors) and BMP inhibitors as described herein may be more effective than either agent alone in applications designed to inhibit vascular remodeling or bone differentiation, because increasing evidence suggests both pathways function cooperatively to effect cell differentiation, and vascular cell migration (Kluppel et al. Bioessays 27:115-118, 2005). These therapies may be synergistic in the treatment of tumors in which one or both pathways is deranged (Katoh, Stem Cell Rev. 3:30-38, 2007).

Combined use of an Indian Hedgehog (IHH) antagonist and a BMP inhibitor as described herein may inhibit pathologic bone formation. IHH is responsible for the commitment of bone precursors to chondrocyte or cartilage forming cells. Endochondral bone formation involves coordinated activity of both chondrogenesis (promoted by BMP signals and IHH signals) and their subsequent calcification by mineralization programs initiated by BMP signals (Seki et al. J. Biol. Chem. 279:18544-18549, 2004; Minina et al. Development 128:4523-4534, 2001). Coadministration of an IHH antagonist with a BMP inhibitor as described herein, therefore, may be more effective in inhibiting pathological bone growth due to hyperactive BMP signaling (such as in FOP), or in any of the inflammatory or traumatic disorders of pathologic bone formation described above.

Strong experimental evidence exists for an effect of both Smo antagonism and BMP antagonism for treating glioblastoma. Compounds as described herein may be used in combination with Smo antagonists to treat glioblastoma.

R. Inhibition of BMP Signaling in Insects

Some of the compounds as described herein may have activity against, and perhaps even selectivity for the BMP receptors of arthropods versus those of chordates. Inhibiting BMP signaling in arthropod larvae or eggs is likely to cause severe developmental abnormalities and perhaps compromise their ability to reproduce, e.g., via the same dorsalization that is observed in zebrafish and drosophila when this pathway is inhibited. If BMP inhibitors as described herein have very strong selectivity for arthropod BMP receptors versus those of humans, they may be used as insecticides or pest control agents that are demonstrably less toxic or more environmentally sound than current strategies.

In addition to being administered to patients in therapeutic methods, compounds as described herein can also be used to treat cells and tissues, as well as structural materials to be implanted into patients (see above), ex vivo. For example, the compounds can be used to treat explanted tissues that may be used, for example, in transplantation.

The invention now being generally described, it will be more readily understood by reference to the following examples which are included merely for purposes of illustration of certain aspects and embodiments of the present invention, and are not intended to limit the invention.

### EXEMPLIFICATION

The synthesis and in vitro and in vivo evaluation of certain BMP inhibitors disclosed herein is set forth in WO 2009/114180, which is herein incorporated by reference in its entirety.

### Example 1

### Synthetic Protocols

Chemistry Material and Methods. Unless otherwise noted, all reagents and solvents were purchased from commercial sources and used without further purification. The NMR spectra were obtained using a 300 or 500 MHz spectrometer. All ¹H NMR spectra are reported in δ units (ppm) and were recorded in CDCl₃ and referenced to the peak for tetramethylsilane (TMS) or in DMSO. Coupling constants (J) are reported in hertz. Column chromatography was performed utilizing a CombiFlash Sg 100c separation system with RediSep disposable silica gel columns. High-resolution mass spectra were obtained by using AccuTOF with a DART source. All test compounds reported in this manuscript had a purity ≧95% as determined by high-performance liquid chromatography (HPLC) analyses using an instrument equipped with a quaternary pump and a SB-C8 column (30×4.6 mm, 3.5 μm). UV absorption was monitored at λ=254 nm. The injection volume was 5 μL. HPLC gradient went from 5% acetonitrile/95% water to 95% acetonitrile/5% water (both solvents contain 0.1% trifluoroacetic acid) over 1.9 min with a total run time of 3.0 min and a flow rate of 3.0 mL/min.

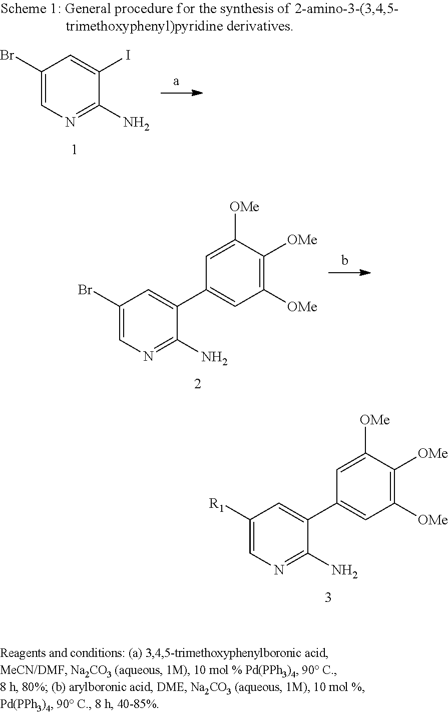

Synthesis of 2-amino-5-bromo-3-(3,4,5-trimethoxyphenyl)pyridine (2). A mixture of 5-bromo-3-iodopyridin-2-amine (386 mg, 1.30 mmol), 3,4,5-trimethoxyphenylboronic acid (275 mg, 1.30 mmol) and Pd(PPh₃)₄ (180 mg, 0.156 mmol) were added to a sealed tube. The tube was evacuated and backfilled with argon (3 cycles). Acetonitrile (6.0 mL) and DMF (2.5 mL) were added by syringe at room temperature, followed by (1M) aqueous Na₂CO₃ (2.6 mL, 2.60 mmol). After being stirred at 90° C. for about 8 h, the reaction mixture was filtered and concentrated. The residue was purified by flash column chromatography to give 2 as white solid (235 mg, 80%). ¹HNMR (500 MHz, CDCl₃) δ8.11 (d, J=2.5Hz, 1H), 7.48 (d, J=2.5Hz, 1H), 6.62 (s, 2H), 3.90 (s, 3H), 3.88 (s, 6H); MS (ESI): 339.0 [M]⁺.
General synthesis of 2-amino-5-aryl-3-(3,4,5-trimethoxyphenyl)pyridines (3). To a solution of 2 (1.0 equiv), an aryl boronic acid (1.1 equiv) and Pd(PPh₃)₄ (0.12 equiv) in DME, (1M) aqueous Na₂CO₃ (2.0 equiv) was added. The reaction mixture was stirred under an argon atmosphere at 90° C. for 8 h. The reaction mixture was filtered and then concentrated. The residue was purified by flash column chromatography, eluting with a mixture of cyclohexane and EtOAc to give products 3.
3-(6-Amino-5-(3,4,5-trimethoxyphenyl)pyridin-3-yl)phenol (K02288): Yield: 40%. ¹HNMR (500 MHz, CDCl₃) δ8.48 (d, J=2.0Hz, 1H), 7.69 (d, J=2.0Hz, 1H), 7.34 (t, J=7.5Hz, 1H), 7.20 (d, J=2.0Hz, 1H), 7.08 (d, J=8.0Hz, 1H), 6.90 (dd, J=2.0, 7.0Hz, 1H), 6.68 (s, 2H), 4.81 (br, 2H), 3.91 (s, 3H), 3.89 (s, 6H); HRMS (ESI) calcd for C₂₀H₂₁N₂O₄ 353.1501 [M+H]⁺; found 353.1462, purity 95.6% (t_{R} 1.35 min).
4-(6-Amino-5-(3,4,5-trimethoxyphenyl)pyridin-3-yl)phenol (11): Yield: 42%. ¹HNMR (500 MHz, CDCl₃) δ8.27 (d, J=2.5Hz, 1H), 7.57 (d, J=2.5Hz, 1H), 7.43-7.41 (m, 2H), 6.92-6.90 (m, 2H), 6.90 (dd, J=2.0, 7.0Hz, 1H), 6.69 (s, 2H), 4.64 (br, 2H), 3.91 (s, 3H), 3.89 (s, 6H); HRMS (ESI) calcd for C₂₀H₂₁N₂O₄ 353.1501 [M+H]⁺; found 353.1490, purity 100.0% (t_{R} 1.32 min).
4-(6-amino-5-(3,4,5-trimethoxyphenyl)pyridin-3-yl)-2-methoxyphenol (12): Yield: 70%. ¹HNMR (500 MHz, CDCl₃) δ8.27 (d, J=2.5 Hz, 1H), 7.56 (d, J=2.5 Hz, 1H), 7.06-6.98 (m, 3H), 6.70 (s, 2H), 4.65 (br, 2H), 3.95 (s, 3H), 3.91 (s, 3H), 3.89 (s, 6H); HRMS (ESI) calcd for C₂₁H₂₃N₂O₅ 383.1607 [M+H]⁺; found 383.1603, purity 98.3% (t_{R} 1.34 min).
N-(3-(6-amino-5-(3,4,5-trimethoxyphenyl)pyridin-3-yl)phenyl)methanesulfonamide (13): Yield: 85%. ¹HNMR (500 MHz, CDCl₃) δ8.89 (br, 1H), 8.39 (d, J=2.5Hz, 1H), 7.61 (d, J=1.5Hz, 1H), 7.47-7.40 (m, 3H), 7.34 (dt, J=1.5, 7.5Hz, 1H), 6.68 (s, 2H), 5.19 (br, 2H), 3.91 (s, 3H), 3.90 (s, 6H) 3.06 (s, 3H); HRMS (ESI) calcd for C₂₁H₂₄N₃O₅S 430.1437 [M+H]⁺; found 430.1412, purity 99.3% (t_{R} 1.34 min).

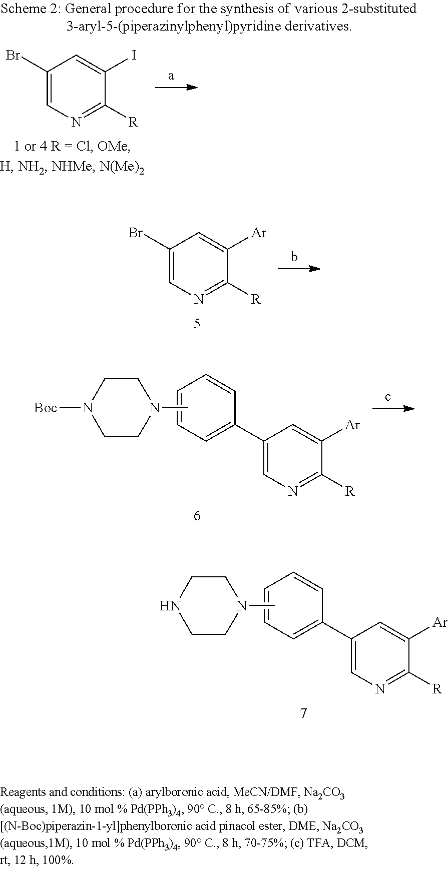

General synthesis of 3-aryl-5-bromopyridines (5). A mixture of 5-bromo-3-iodopyridin-2-amine (1.0 equiv), arylboronic acid (1.0 equiv), Pd(PPh₃)₄ (0.12 equiv) were added to a sealed tube. The tube was evacuated and backfilled with argon (3 cycles). Acetonitrile and DMF (3:1 mL) were added by syringe at room temperature, followed by (1M) aqueous Na₂CO₃ (2.0 equiv). After being stirred at 90° C. for about 8 h, the reaction mixture was filtered and concentrated. The residue was purified by flash column chromatography to give 5.
General synthesis of 3-aryl-5-((N-Boc)-piperazinylphenyl)pyridines (6). To a solution of 5 (1.0 equiv), [(N-Boc)piperazin-1-yl]phenylboronic acid pinacol ester (1.1 equiv) and Pd(PPh₃)₄ (0.12 equiv) in DME, (1M) aqueous Na₂CO₃ (2.0 equiv) was added. The reaction mixture was stirred under argon atmosphere at 90° C. for 8 h. The reaction mixture was filtered and concentrated. The residue was purified by flash column chromatography, eluting with a mixture cyclohexane/EtOAc to give 6.
General synthesis of 3-aryl-5-(piperazinylphenyl)pyridines (7). To a stirring solution of the 6 (0.01 mmol) in dry CH₂C₂(2 mL) at 0° C., trifluoroacetic acid (0.2 mL) was slowly added and the reaction mixture was stirred overnight at room temperature. The mixture was concentrated under vacuum. The residue was suspected in ethyl acetate (10 mL) and then a saturated NaHCO₃ solution was added to adjust the pH to 7 at 0° C. The mixture was extracted with ethyl acetate (3×10 mL). The combined organic layer was dried over anhydrous Na₂SO₄, filtered and concentrated in vacuo. The remaining residue was subjected to column chromatography to furnish 7 as a white to light yellow foam.
5-(3-(piperazin-1-yl)phenyl)-3-(3,4,5-trimethoxyphenyl)pyridin-2-amine (14): Yield: 82%. ¹HNMR (500 MHz, CDCl₃) δ8.31 (d, J=2.5 Hz, 1H), 7.61 (d, J=2.5 Hz, 1H), 7.35 (d, J=8.0 Hz, 1H), 7.07 (t, J=2.0 Hz, 1H), 7.04-7.03 (m, 1H), 6.92-6.90 (m, 1H), 6.70 (s, 2H), 4.68 (br, 2H), 3.91 (s, 3H), 3.89 (s, 6H), 3.21-3.19 (m, 4H), 3.06-3.04 (m, 4H); HRMS (ESI) calcd for C₂₄H₂₈N₄O₃ 421.2240 [M+H]⁺; found 421.2215, purity 98.7% (t_{R} 1.12 min).
5-(4-(piperazin-1-yl)phenyl)-3-(3,4,5-trimethoxyphenyl)pyridin-2-amine (15): Yield: 77%. ¹HNMR (500 MHz, CDCl₃) δ8.29 (d, J=2.0 Hz, 1H), 7.58 (d, J=2.5 Hz, 1H), 7.47-7.45 (m, 2H), 7.00-6.98 (m, 2H), 6.70 (s, 2H), 4.61 (br, 2H), 3.91 (s, 3H), 3.89 (s, 6H), 3.26-3.24 (m, 0.6H) and 3.20-3.18 (m, 3.4H) due to rotamer, 3.07-3.05 (m, 3.4H) and 2.72-2.70 (m, 0.6H) due to rotamer; HRMS (ESI) calcd for C₂₄H₂₈N₄O₃ 421.2240 [M+H]⁺; found 421.2259, purity 98.6% (t_{R} 1.05 min).
3-(3,4-dimethoxyphenyl)-5-(4-(piperazin-1-yl)phenyl)pyridin-2-amine (16): Yield: 80%. ¹HNMR (500 MHz, CDCl₃) 8.28 (d, J=2.5 Hz, 1H), 7.57 (d, J=2.0 Hz, 1H), 7.47-7.45 (m, 2H), 7.06-7.04 (m, 1H), 7.01-6.97 (m, 4H), 4.58 (br, 2H), 3.94 (s, 3H), 3.91 (s, 3H), 3.26-3.24 (m, 0.3H) and 3.20-3.18 (m, 3.7H) due to rotamer, 3.07-3.05 (m, 3.7H) and 2.72-2.70 (m, 0.3H) due to rotamer; HRMS (ESI) calcd for C₂₃H₂₇N₄O₂ 391.2134 [M+H]⁺; found 391.2142, purity 97.9% (t_{R} 1.08 min).
3-(3,5-dimethoxyphenyl)-5-(4-(piperazin-1-yl)phenyl)pyridin-2-amine (17): Yield: 85%. ¹HNMR (500 MHz, CDCl₃) δ8.27 (d, J=2.5 Hz, 1H), 7.59 (d, J=2.5 Hz, 1H), 7.46-7.44 (m, 2H), 7.00-6.98 (m, 2H), 6.63 (d, J=2.0 Hz, 2H), 6.50 (t, J=2.5 Hz, 1H), 4.76 (br, 2H), 3.83 (s, 6H), 3.21-3.19 (m, 4H), 3.08-3.06 (m, 4H); HRMS (ESI) calcd for C₂₃H₂₇N₄O₂ 391.2134 [M+H]⁺; found 391.2159, purity 97.7% (t_{R} 1.16 min).
3-(3-methoxyphenyl)-5-(4-(piperazin-1-yl)phenyl)pyridin-2-amine (18): Yield: 82%. ¹HNMR (500 MHz, CDCl₃) δ8.28 (d, J=2.0 Hz, 1H), 7.59 (d, J=2.0 Hz, 1H), 7.46-7.44 (m, 2H), 7.41 (t, J=8.0 Hz, 1H), 7.09-7.07 (m, 1H), 7.03 (t, J=2.0 Hz, 1H), 7.00-6.98 (m, 2H), 6.95 (dd, J=2.0, 8.5 Hz, 1H), 4.69 (br, 2H), 3.85 (s, 3H), 3.26-3.24 (m, 0.7H) and 3.22-3.20 (m, 3.3H) due to rotamer, 3.09-3.07 (m, 3.3H) and 2.72-2.70 (m, 0.7H) due to rotamer; HRMS (ESI) calcd for C₂₂H₂₅N₄O 361.2028 [M+H]⁺; found 361.2043, purity 97.5% (t_{R} 1.16 min).
3-(2,3-dihydrobenzo[b][1,4]dioxin-6-yl)-5-(4-(piperazin-1-yl)phenyl)pyridin-2-amine (19): Yield: 80%. ¹HNMR (500 MHz, CDCl₃) δ8.25 (d, J=2.5 Hz, 1H), 7.54 (d, J=2.0 Hz, 1H), 7.45-7.43 (m, 2H), 7.02-7.01 (m, 1H), 6.99-6.96 (m, 4H), 4.63 (br, 2H), 4.31 (s, 4H), 3.25-3.23 (m, 0.8H) and 3.20-3.18 (m, 3.2H) due to rotamer, 3.07-3.05 (m, 3.2H) and 2.72-2.70 (m, 0.8H) due to rotamer; HRMS (ESI) calcd for C₂₃H₂₅N₄O₂ 389.1978 [M+]⁺; found 389.2003, purity 97.0% (t_{R} 1.16 min).
3-(4-methoxyphenyl)-5-(4-(piperazin-1-yl)phenyl)pyridin-2-amine (20): Yield: 78%. ¹HNMR (500 M Hz, CDCl₃) δ8.27 (d, J=2.5 Hz, 1H), 7.55 (d, J=2.0 Hz, 1H), 7.46-7.42 (m, 4H), 7.02-6.98 (m, 4H), 4.55 (br, 2H), 3.87 (s, 3H), 3.19-3.18 (m, 4H), 3.07-3.05 (m, 4H); HRMS (ESI) calcd for C₂₂H₂₅N₄O 361.2028 [M+]⁺; found 361.2055, purity 97.7% (t_{R} 1.20 min).
3-(3-isopropoxyphenyl)-5-(4-(piperazin-1-yl)phenyl)pyridin-2-amine (21): Yield: 80%. ¹HNMR (300 M Hz, CDCl₃) δ8.29 (d, J=2.4 Hz, 1H), 7.58 (d, J=2.4 Hz, 1H), 7.47-7.44 (m, 2H), 7.39 (d, J=8.4 Hz, 1H), 7.06-6.97 (m, 4H), 6.93-6.89 (m, 1H), 4.63-4.55 (m, 3H), 3.20-3.17 (m, 4H), 3.07-3.04 (m, 4H), 1.37 (d, J=6.0 Hz, 6H); HRMS (ESI) calcd for C₂₄H₂₉N₄O 389.2341 [M+]⁺; found 389.2362, purity 100.0% (t_{R} 1.16 min).
3-(4-chloro-3-methoxyphenyl)-5-(4-(piperazin-1-yl)phenyl)pyridin-2-amine (22): Yield: 82%. ¹HNMR (300 MHz, CDCl₃) δ8.27 (d, J=2.1 Hz, 1H), 7.57 (d, J=2.4 Hz, 1H), 7.48-7.44 (m, 3H), 7.04-6.98 (m, 4H), 4.83 (br, 2H), 3.94 (s, 3H), 3.32-3.29 (m, 3.6H) and 3.26-3.22 (m, 0.4H) due to rotamer, 3.18-3.15 (m, 3.6H) and 2.74-2.69 (m, 0.4H) due to rotamer; HRMS (ESI) calcd for C₂₂H₂₄ClN₄O 395.1639 [M+]⁺; found 395.1647, purity 96.6% (t_{R} 1.18 min).
3-(3-methoxy-4-methylphenyl)-5-(4-(piperazin-1-yl)phenyl)pyridin-2-amine (23): Yield: 84%. ¹HNMR (300 MHz, CDCl₃) δ8.28 (d, J=2.4 Hz, 1H), 7.58 (d, J=2.4 Hz, 1H), 7.47-7.44 (m, 2H), 7.24-7.21 (m, 1H), 7.00-6.97 (m, 3H), 6.93 (d, J=1.5 Hz, 1H), 4.65 (br, 2H), 3.85 (s, 3H), 3.20-3.16 (m, 4H), 3.07-3.03 (m, 4H), 2.67 (s, 3H); HRMS (ESI) calcd for C₂₃H₂₇N₄O 375.2185 [M+]⁺; found 375.2189, purity 100.0% (t_{R} 1.16 min).
N-methyl-5-(4-(piperazin-1-yl)phenyl)-3-(3,4,5-trimethoxyphenyl)pyridin-2-amine (24): Yield: 92%. ¹HNMR (500 MHz, CDCl₃) δ8.39 (d, J=2.0 Hz, 1H), 7.50 (d, J=2.5 Hz, 1H), 7.46-7.45 (m, 2H), 7.00-6.98 (m, 2H), 6.63 (s, 2H), 4.67 (q, J=5.0 Hz, 1H), 3.91 (s, 3H), 3.88 (s, 6H), 3.25-3.22 (m, 0.6H) and 3.20-3.18 (m, 3.4H) due to rotamer, 3.08-3.06 (m, 3.6H) and 2.72-2.70 (m, 0.4H) due to rotamer, 3.01 (d, J=5.0 Hz, 3H); HRMS (ESI) calcd for C₂₅H₃₁N₄O₃ 435.2396 [M+]⁺; found 5435.2396, purity 98.9% (t_{R} 1.10 min).
N,N-dimethyl-5-(4-(piperazin-1-yl)phenyl)-3-(3,4,5-trimethoxyphenyl)pyridin-2-amine (25): Yield: 90%. ¹HNMR (500 MHz, CDCl₃) δ8.40 (d, J=2.0 Hz, 1H), 7.62 (d, J=2.5 Hz, 1H), 7.48-7.46 (min, 2H), 7.00-6.99 (m, 2H), 6.76 (s, 2H), 3.90 (s, 3H), 3.88 (s, 6H), 3.21-3.19 (m, 4H), 3.07-3.05 (m, 4H), 2.78 (s, 6H); HRMS (ESI) calcd for C₂₆H₃₃N₄O₃ 449.2553 [M+]⁺; found 449.2575, purity 97.8% (t_{R} 1.14 min).
1-(4-(5-(3,4,5-trimethoxyphenyl)pyridin-3-yl)phenyl)piperazine (26): Yield: 95%. ¹HNMR (500 MHz, CDCl₃) δ8.78 (d, J=2.0 Hz, 1H), 8.71 (d, J=2.5 Hz, 1H), 7.95 (t, J=2.5 Hz, 1H), 7.58-7.56 (m, 2H), 7.06-7.04 (m, 2H), 6.80 (s, 2H), 3.95 (s, 6H), 3.91 (s, 3H), 3.25-3.23 (m, 4H), 3.08-3.06 (m, 4H); HRMS (ESI) calcd for C₂₄H₂₈N₃O₃ 406.2131 [M+]⁺; found 406.2142, purity 100.0% (t_{R} 1.20 min).
1-(4-(6-chloro-5-(3,4,5-trimethoxyphenyl)pyridin-3-yl)phenyl)piperazine (27): Yield: 94%. ¹HNMR (300 MHz, CDCl₃) δ8.57 (d, J=2.4 Hz, 1H), 7.83 (d, J=2.7 Hz, 1H), 7.53-7.50 (m, 2H), 7.03-7.00 (m, 2H), 6.70 (s, 2H), 3.92 (s, 3H), 3.90 (s, 6H), 3.32-3.28 (m, 0.5H) and 3.27-3.24 (m, 3.5H) due to rotamer, 3.10-3.07 (m, 3.5H) and 2.75-2.69 (m, 0.5H) due to rotamer; HRMS (ESI) calcd for C₂₄H₂₇ClN₃O₃ 440.1741 [M+]⁺; found 440.1723, purity 95.6% (t_{R} 1.42 min).
1-(4-(6-methoxy-5-(3,4,5-trimethoxyphenyl)pyridin-3-yl)phenyl)piperazine (28): Yield: 79%. ¹HNMR (500 MHz, CDCl₃) δ8.34 (d, J=2.0 Hz, 1H), 7.78 (d, J=2.5 Hz, 1H), 7.50-7.48 (m, 2H), 7.03-7.01 (m, 2H), 6.81 (s, 2H), 4.02 (s, 3H), 3.90 (s, 9H), 3.28-3.26 (m, 0.3H) and 3.23-3.21 (m, 3.7H) due to rotamer, 3.08-3.07 (m, 3.7H) and 2.73-2.71 (m, 0.3H) due to rotamer; HRMS (ESI) calcd for C₂₅H₂₉N₃O₄ 436.2236 [M+]⁺; found 436.2265, purity 100.0% (t_{R} 1.38 min).
5-(4-(piperazin-1-yl)phenyl)phenyl)-3-(quinolin-4-yl)pyridin-2-amine (29): Yield: 79%. ¹HNMR (500 MHz, CDCl₃) δ9.02 (d, J=4.5 Hz, 1H), 8.46 (d, J=2.5 Hz, 1H), 8.22 (d, J=8.0 Hz, 1H), 7.79-7.75 (m, 2H), 7.64 (d, J=2.5 Hz, 1H), 7.57-7.53 (m, 1H), 7.48-7.43 (m, 3H), 7.01-6.98 (m, 2H), 4.34 (br, 2H), 3.25-3.23 (m, 0.6H) and 3.20-3.18 (m, 3.4H) due to rotamer, 3.07-3.05 (m, 3.4H) and 2.72-2.70 (m, 0.6H) due to rotamer; HRMS (ESI) calcd for C₂₄H₂₄N₅ 382.2032 [M+]⁺; found 382.1993, purity 97.7% (t_{R} 1.04 min).
5-(3-(piperazin-1-yl)phenyl)-3-(quinolin-4-yl)pyridin-2-amine (30): Yield: 85%. ¹HNMR (500 MHz, CDCl₃) δ9.03 (d, J=4.0 Hz, 1H), 8.48 (s, 1H), 8.22 (d, J=8.0 Hz, 1H), 7.80-7.74 (m, 2H), 7.66 (s, 1H), 7.57 (d, J=8.0 Hz, 1H), 7.47-7.44 (m, 1H), 7.35 (t, J=8.0 Hz, 1H), 7.07-7.03 (m, 2H), 6.92 (d, J=8.5 Hz, 1H), 4.38 (br, 2H), 3.26-3.22 (m, 1H) and 3.21-3.19 (m, 3H) due to rotamer, 3.05-3.04 (m, 3H) and 2.71-2.66 (m, 1H) due to rotamer; HRMS (ESI) calcd for C₂₄H₂₄N₅ 382.2032 [M+]⁺; found 382.2024, purity 95.1% (t_{R} 1.09 min).
5-(4-(piperazin-1-yl)phenyl)-3-(quinolin-5-yl)pyridin-2-amine (31): Yield: 84%. ¹HNMR (500 MHz, CDCl₃) δ8.98 (dd, J=2.0, 4.5 Hz, 1H), 8.44 (d, J=2.5 Hz, 1H), 8.21 (d, J=9.0 Hz, 1H), 8.06-8.04 (m, 1H), 7.84-7.81 (m, 1H), 7.63 (d, J=2.5 Hz, 1H), 7.59-7.58 (m, 1H), 7.48-7.46 (m, 2H), 7.41-7.39 (m, 1H), 7.00-6.97 (m, 2H), 4.29 (br, 2H), 3.25-3.23 (m, 0.4H) and 3.20-3.18 (m, 3.6H) due to rotamer, 3.06-3.04 (m, 3.6H) and 2.71-2.69 (m, 0.4H) due to rotamer; HRMS (ESI) calcd for C₂₄H₂₄N₅ 382.2032 [M+]⁺; found 382.2039, purity 95.2% (t_{R} 0.97 min).
1-(4-(5-(3,5-dimethoxyphenyl)pyridin-3-yl)phenyl)piperazine (33): Yield: 98%. ¹HNMR (300 MHz, CDCl₃) δ8.79 (d, J=2.4 Hz, 1H), 8.73 (d, J=2.4 Hz, 1H), 7.99 (t, J=2.1 Hz, 1H), 7.57-7.54 (m, 2H), 7.04-7.02 (m, 2H), 6.76 (d, J=. 1.8 Hz, 2H), 6.53 (t, J=2.1 Hz, 1H), 3.86 (s, 6H), 3.23-3.21 (m, 4H), 3.06-3.04 (m, 4H); HRMS (ESI) calcd for C₂₃H₂₆N₃O₂ 376.2025 [M+]⁺; found 376.2023, purity 100.0% (t_{R} 1.26 min).

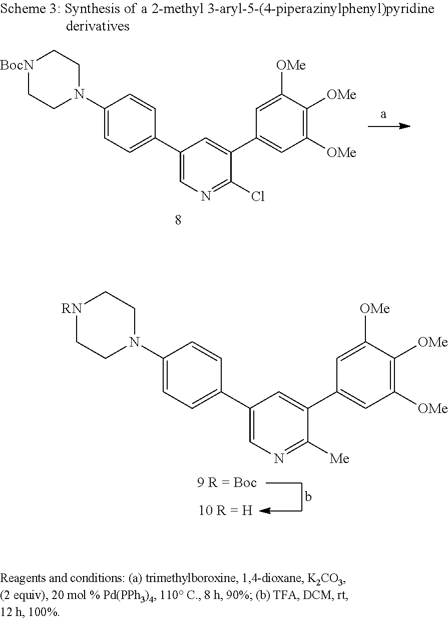

Synthesis of 1-(4-(6-methyl-5-(3,4,5-trimethoxyphenyl)pyridin-3-yl)phenyl)piperazine (10): A mixture of N-Boc-4-(4-(6-chloro-5-(3,4,5-trimethoxyphenyl)pyridin-3-yl)phenyl)piperazine (43 mg, 0.080 mmol), trimethylboroxine (46 μL, 0.32 mmol), Pd(PPh₃)₄ (19 mg, 0.016 mmol) and K₂CO₃ (22 mg, 0.16 mmol) were added to a sealed tube. The tube was evacuated and backfilled with argon (3 cycles). 1,4-Dioxane (1.0 mL) was added by syringe at room temperature. After being stirred at 110° C. for 8 h, the reaction mixture was filtered and concentrated. The residue purified by flash column chromatography to give 9 (40 mg, 96%). ¹HNMR (300 MHz, CDCl₃) δ8.70 (d, J=2.4 Hz, 1H), 7.69 (d, J=2.4 Hz, 1H), 7.54-7.51 (m, 2H), 7.02-6.99 (m, 2H), 6.55 (s, 2H), 3.91 (s, 3H), 3.88 (s, 6H), 3.61-3.58 (m, 4H), 3.21-3.18 (m, 4H), 2.55 (s, 3H), 1.48 (s, 9H); MS (ESI): 519.5 [M]⁺. The carbamate protecting group of 9 (40 mg) was removed using the general method previously described using TFA to furnish 10 as a white foam (30 mg, 93%). ¹HNMR (300 MHz, CDCl₃) δ8.71 (d, J=2.1 Hz, 1H), 7.70 (d, J=2.4 Hz, 1H), 7.55-7.52 (m, 2H), 7.03-7.00 (m, 2H), 6.56 (s, 2H), 3.92 (s, 3H), 3.89 (s, 6H), 3.24-3.20 (m, 4H), 3.08-3.05 (m, 4H), 2.55 (s, 3H); HRMS (ESI) calcd for C₂₅H₃₀N₃O₃ 420.2287 [M+]⁺; found 420.2295, purity 95.5% (t_{R} 1.13 min).
Synthesis of 3-(3,4,5-trimethoxyphenyl)-6-[4-(1-piperazinyl)phenyl]pyrazolo[1,5-a]pyrimidine (32): This compound was prepared using the reported methodology of Cuny, G. D.; Yu, P. B.; Laha, J. K.; Xing, X.; Liu, J. F.; Lai, C. S.; Deng, D. Y.; Sachidanandan, C.; Bloch, K. D.; Peterson, R. T. Structure-activity relationship study of bone morphogenetic protein (BMP) signaling inhibitors. Bioorg Med Chem Lett 2008, 18, 4388-92. ¹HNMR (500 MHz, DMSO) δ9.38 (d, J=2.0 Hz, 1H), 9.04 (d, J=2.0 Hz, 1H), 8.80 (s, 1H), 7.75 (d, J=9.0 Hz, 2H), 7.51 (s, 2H), 7.07 (d, J=9.0 Hz, 1H), 3.88 (s, 6H), 3.70 (s, 3H), 3.25-3.18 (m, 4H), 2.92-2.90 (m, 4H); FIRMS (ESI) calcd for C₂₅H₂₇N₅O₃ 446.2192 [M+]⁺; found 446.2186, purity 100% (t_{R} 1.43 min).

### Example 2

### Representative Compounds

TABLE 1
Representative compounds

| Compd | Structure |
|---|---|
| K02288a | 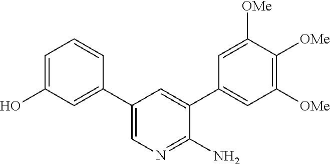 [SMILES: COc1cc(-c2cc(-c3cccc(O)c3)cnc2N)cc(OC)c1OC] |
| 10 | 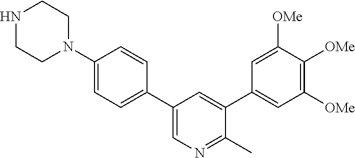 [SMILES: COc1cc(-c2cc(-c3ccc(N4CCNCC4)cc3)cnc2C)cc(OC)c1OC] |
| 11 | 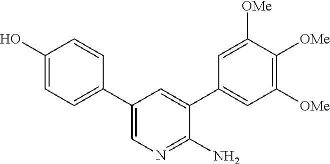 [SMILES: COc1cc(-c2cc(-c3ccc(O)cc3)cnc2N)cc(OC)c1OC] |
| 12 | 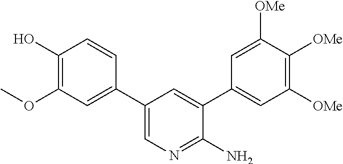 [SMILES: COc1cc(-c2cnc(N)c(-c3cc(OC)c(OC)c(OC)c3)c2)ccc1O] |
| 13 | 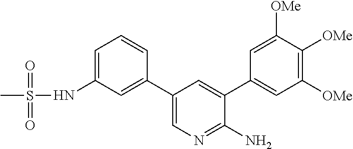 [SMILES: COc1cc(-c2cc(-c3cccc(NS(C)(=O)=O)c3)cnc2N)cc(OC)c1OC] |
| 14 | 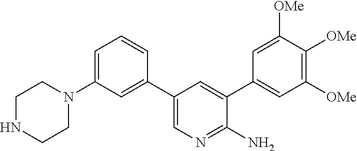 [SMILES: COc1cc(-c2cc(-c3cccc(N4CCNCC4)c3)cnc2N)cc(OC)c1OC] |
| 15 | 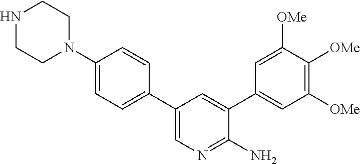 [SMILES: COc1cc(-c2cc(-c3ccc(N4CCNCC4)cc3)cnc2N)cc(OC)c1OC] |
| 16 | 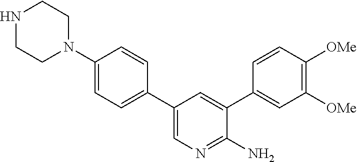 [SMILES: COc1ccc(-c2cc(-c3ccc(N4CCNCC4)cc3)cnc2N)cc1OC] |
| 17 | 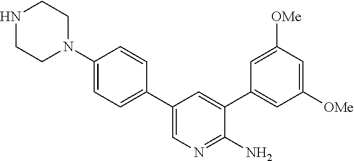 [SMILES: COc1cc(OC)cc(-c2cc(-c3ccc(N4CCNCC4)cc3)cnc2N)c1] |
| 18 | 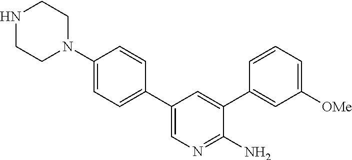 [SMILES: COc1cccc(-c2cc(-c3ccc(N4CCNCC4)cc3)cnc2N)c1] |
| 19 | 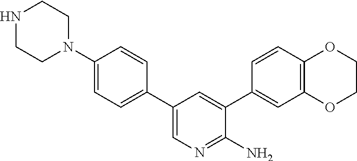 [SMILES: Nc1ncc(-c2ccc(N3CCNCC3)cc2)cc1-c1ccc2c(c1)OCCO2] |
| 20 | 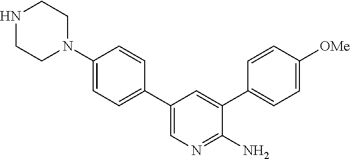 [SMILES: COc1ccc(-c2cc(-c3ccc(N4CCNCC4)cc3)cnc2N)cc1] |
| 21 | 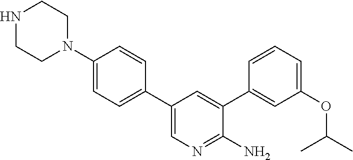 [SMILES: CC(C)Oc1cccc(-c2cc(-c3ccc(N4CCNCC4)cc3)cnc2N)c1] |
| 22 | 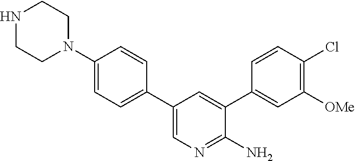 [SMILES: COc1cc(-c2cc(-c3ccc(N4CCNCC4)cc3)cnc2N)ccc1Cl] |
| 23 | 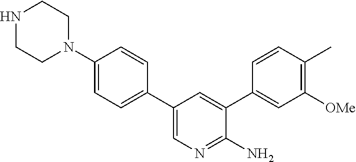 [SMILES: COc1cc(-c2cc(-c3ccc(N4CCNCC4)cc3)cnc2N)ccc1C] |
| 24 | 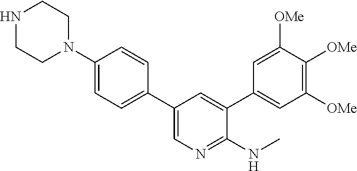 [SMILES: CNc1ncc(-c2ccc(N3CCNCC3)cc2)cc1-c1cc(OC)c(OC)c(OC)c1] |
| 25 | 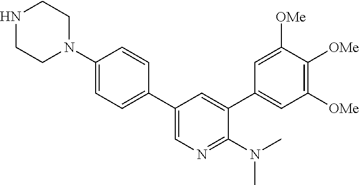 [SMILES: COc1cc(-c2cc(-c3ccc(N4CCNCC4)cc3)cnc2N(C)C)cc(OC)c1OC] |
| 26 | 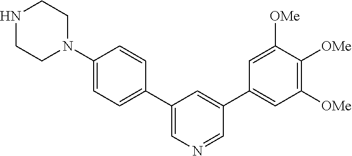 [SMILES: COc1cc(-c2cncc(-c3ccc(N4CCNCC4)cc3)c2)cc(OC)c1OC] |
| 27 | 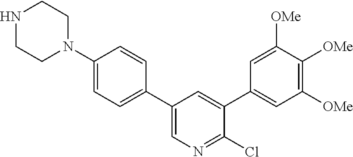 [SMILES: COc1cc(-c2cc(-c3ccc(N4CCNCC4)cc3)cnc2Cl)cc(OC)c1OC] |
| 28 | 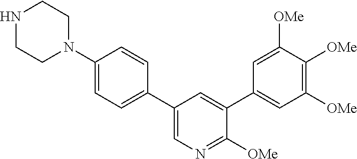 [SMILES: COc1cc(-c2cc(-c3ccc(N4CCNCC4)cc3)cnc2OC)cc(OC)c1OC] |
| 29 | 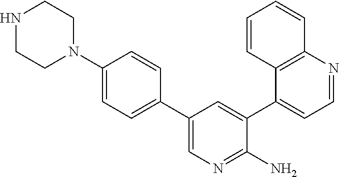 [SMILES: Nc1ncc(-c2ccc(N3CCNCC3)cc2)cc1-c1ccnc2ccccc12] |
| 30 | 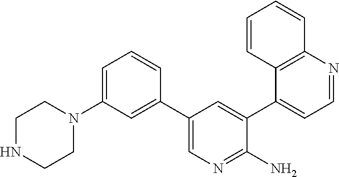 [SMILES: Nc1ncc(-c2cccc(N3CCNCC3)c2)cc1-c1ccnc2ccccc12] |
| 31 | 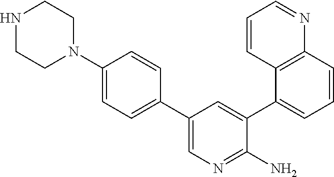 [SMILES: Nc1ncc(-c2ccc(N3CCNCC3)cc2)cc1-c1cccc2ncccc12] |
| 32 | 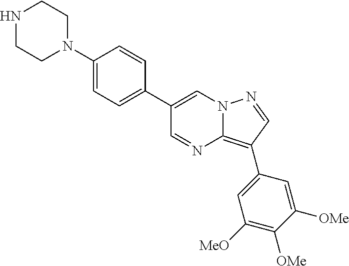 [SMILES: COc1cc(-c2cnn3cc(-c4ccc(N5CCNCC5)cc4)cnc23)cc(OC)c1OC] |
| 33 | 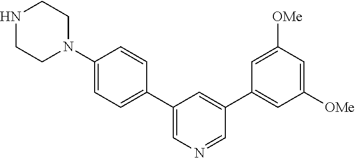 [SMILES: COc1cc(OC)cc(-c2cncc(-c3ccc(N4CCNCC4)cc3)c2)c1] |

### Example 3

### Thermal Shift Kinase Assay

Thermal melting experiments were performed using a Real Time PCR machine Mx3005p (Stratagene) with a protein concentration of 1-2 μM and 10 μM inhibitor as described by Niesen et al., Nat Protoc 2007, 2, 2212-21. Recombinant human kinases for DSF screening were prepared by SGC using the published methods of Sanvitale et al., PLoS One 2013, 8, e62721. The potency and selectivity of certain compounds of the invention based on thermal shift kinase and ligand induced transcriptional activity assays are shown in Table 2.

TABLE 2
Thermal shift and cell-based signaling inhibition results

| | ALK2 | ALK5 | | ALK2 | ALK5 | BMP6 | TGFβ1 | |
|---|---|---|---|---|---|---|---|---|
| | ΔTm | ΔTm | | IC50 | IC50 | IC50 | IC50 | |
| Compound | (° C.) | (° C.) | ΔTmDiff. | (nM) | (nM) | (nM) | (nM) | Fold Select. |
| K02288 | 13.2 | 11.2 | 2.0 | 35 | 280 | 420 ± 170 | 3,400 ± 500 | 8 |
| 11 | 13.5 | 12.0 | 1.5 | nd | nd | 20 ± 1 | 580 ± 50 | 28 |
| 12 | 13.9 | 12.2 | 1.7 | nd | nd | 90 ± 30 | 2,300 ± 300 | 28 |
| 13 | 14.4 | 13.4 | 0.9 | 6 | 180 | 60 ± 10 | 260 ± 20 | 4 |
| 14 | 14.5 | 13.7 | 0.8 | 17 | 49 | 6 ± 1 | 110 ± 20 | 17 |
| 15 | 15.1 | 13.9 | 1.2 | 10 | 186 | 4 ± 1 | 100 ± 10 | 23 |
| 16 | 11.5 | 7.2 | 4.3 | 23 | 6,900 | [symbol] 40 ± 30 | 13,100 ± 1,000 | 92 |
| 17 | 13.9 | 10.4 | 3.5 | 14 | 1,000 | 40 ± 10 | 650 ± 80 | 18 |
| 18 | 12.1 | 8.3 | 3.8 | 86 | 12,300 | [symbol] 10 ± 10 | 3,800 ± 200 | 33 |
| 19 | 9.5 | 5.5 | 4.0 | 1,870 | 15,000 | 730 ± 100 | 38,000 ± 8,500 | 53 |
| 20 | 8.6 | 4.2 | 4.5 | nd | nd | 1,900 ± 900 | 95,000 ± 23,000 | 48 |
| 21 | 8.5 | 7.6 | 1.0 | 790 | 1,400 | 2,500 ± 90 | 2,600 ± 400 | 1 |
| 22 | 11.9 | 9.2 | 2.7 | 63 | 1,910 | 160 ± 10 | 5,800 ± 600 | 16 |
| 23 | 11.2 | 7.3 | 3.8 | 110 | 21,000 | 840 ± 120 | 35,500 ± 13,000 | 42 |
| 24 | 0.3 | 0.9 | −0.5 | nd | nd | [symbol] 80 ± 60 | 28,000 ± 5,300 | 99 |
| 25 | 0.6 | 0.5 | 0.1 | nd | nd | 1,700 ± 400 | 15,000 ± 30,000 | 68 |
| 26 | 14.l | 10.4 | 3.7 | 15 | 240 | 30 ± 2 | 1,300 ± 200 | 51 |
| 27 | 12.8 | 9.0 | 3.8 | nd | nd | 170 ± 60 | 90,000 ± 34,000 | 244 |
| 10 | 13.7 | 9.7 | 4.0 | 24 | 3,000 | 100 ± 1 | 16,000 ± 4,000 | 164 |
| 28 | 1.1 | 1.2 | 0.1 | nd | nd | 1,700 ± 300 | 82,000 ± 1,200 | 48 |
| 29 | 10.4 | 6.9 | 3.4 | 120 | 21,000 | 110 ± 50 | 4,300 ± 300 | 11 |
| 30 | 9.9 | 7.4 | 2.5 | 110 | 5,000 | [symbol] 20 ± 60 | 2,800 ± 300 | 5 |
| 31 | 9.4 | 5.3 | 4.2 | 270 | 99,000 | 170 ± 60 | 34,800 ± 9,000 | 75 |
| 32 | 14.2 | 11.6 | 2.6 | 10 | 30 | 20 ± 2 | 760 ± 80 | 41 |
| 33 | 12.8 | 8.1 | 4.7 | nd | nd | [symbol] 60 ± 40 | 26,000 ± 4,000 | 102 |

[symbol]

indicates data missing or illegible when filed

### Example 4

### Protein Expression and Purification

The human ALK2 kinase domain, residues 201-499 including the activating mutation Q207D, was subcloned into the vector pFB-LIC-Bse. Baculoviral expression was performed in Sf9 insect cells at 27° C., shaking at 110 rpm. Cells were harvested at 72 hours post infection and resuspended in 50 mM HEPES pH 7.5, 500 mM NaCl, 5 mM imidazole, 5% glycerol, 0.1 mM TCEP, supplemented with protease inhibitor set V (Calbiochem). Cells were lysed using a C5 high pressure homogenizer (Emulsiflex) and the insoluble material excluded by centrifugation at 21,000 rpm. Nucleic acids were removed on a DEAE-cellulose column before purification of the N-terminally His-tagged ALK2 protein by Ni-affinity chromatography. The eluted protein was cleaved with TEV protease and further purified by size exclusion chromatography using a S200 HiLoad 16/60 Superdex column. A final clean up step was performed by reverse purification on a Ni-sepharose column and the purified protein stored at −80° C.

### Example 5

### Luciferase Reporter Assay

C2Cl2 myofibroblasts cells stably transfected with BMP responsive element from the Id promoter fused to luciferase reporter gene (BRE-Luc) were generously provided by Dr. Peter ten Dijke (Leiden University Medical Center, NL) following the methods described by Zilberberg et al., BMC cell biology 2007, 8, 41-50. Human embryonic kidney 293T cells stably transfected with the TGF-β responsive element from the PAI-1 promoter fused to luciferase reporter gene (CAGA-Luc) were generously provided by Dr. Howard Weiner (Brigham and Women's Hospital, Boston, Mass.) following the methods described by Oida et al., PloS one 2011, 6, e18365. C2Cl2 Bre-Luc and 293T CAGA-Luc cells were seeded in DMEM supplemented with 2% FBS at 20,000 cells per well in tissue culture treated 96-well plates (Costar® 3610; Corning). The cells were incubated for 1 h (37° C. and 10% CO₂) and allowed to settle and attach. Compounds of interest or DMSO were diluted in DMEM and added at final compound concentrations of 1 nM to 10 μM. Cells were then incubated for 30 min. Adenovirus expressing constitutively active BMP and TGF-β type I receptors (Ad.caALK1-5), generously provided by Dr. Akiko Hata (University of California at San Francisco), were added to achieve a multiplicity of infection (MOI) of 100. Plates were incubated overnight at 37° C. Cell viability was assayed with an MIT (3-(4,5-dimethylthiazol-2-yl)-2,5-diphenyl tetrazolium bromide) colorimetric assay (Promega) per the manufacturer's instructions. Media was discarded, and firefly luciferase activity was measured (Promega) according to manufacturer's protocol. Light output was measured using a Spectramax L luminometer (Molecular Devices) with an integration time of one second per well. Data was normalized to 100% of incremental BRE-Luc activity due to adenoviruses specifying caALK1, 2, or 3, or the incremental CAGA-Luc activity due to adenoviruses specifying caALK4 or 5. Graphing and regression analysis by sigmoidal dose-response with variable Hill coefficient was performed using GraphPad Prism software.

### Example 6

### Cell Viability Assay

HePG2 hepatocarcinoma cells were seeded in DMEM supplemented with 10% FBS at 25,000 cells per well in tissue culture treated 96-well plates (Costar® 3610; Corning). The cells were incubated for 2 h (37° C. and 5% CO₂) and allowed to settle and attach. Compounds of interest or DMSO were diluted in DMEM and added at final compound concentrations of 1 μM, 10 μM, and 100 μM. Cells were incubated for 4 hours and 24 hours after which the media was discarded. Cells were lysed by adding 30 μL of passive lysis buffer (Promega) and shaken at RT for 15 min. Cell viability was determined by quantifying the ATP present in each well by adding 10 μL of CellTiter-Glo (Promega) per well and measuring the light output Spectramax L luminometer (Molecular Devices) with an integration time of one second per well. Data was normalized to 100% viability for cells receiving only DMSO without any concurrent compound.

Results from the cell viability assay for several compounds of the invention and other currently FDA approved kinase inhibitors are shown in Table 3. In certain instances where multiple tests were performed for a particular compound in a particular assay, the data shown in Table 3 represents an average of the individual results.

TABLE 3
Cell viability results

| | 4 hr | | | 24 hr | | |
|---|---|---|---|---|---|---|
| Name | 1 μM | 10 μM | 100 μM | 1 μM | 10 μM | 100 μM |
| Imatinib | 93% | 99% | 98% | 94% | 72% | 13% |
| Gefitinib | 93% | 105% | 104% | 98% | 82% | 85% |
| Sorafenib | 94% | 98% | 92% | 93% | 79% | 7% |
| Erlotinib | 96% | 107% | 108% | 99% | 84% | 84% |
| Dasatinib | 94% | 107% | 75% | 87% | 75% | 38% |
| Sunitinib | 97% | 107% | 28% | 96% | 68% | 5% |
| Nilotinib | 99% | 106% | 105% | 100% | 101% | 91% |
| Lapatinib | 100% | 104% | 104% | 98% | 92% | 89% |
| Pazopanib | 100% | 103% | 105% | 96% | 90% | 92% |
| Ruxolitinib | 101% | 105% | 88% | 100% | 90% | 55% |
| Crizotinib | 104% | 103% | 8% | 101% | 80% | 5% |
| Vemurafenib | 103% | 100% | 102% | 95% | 77% | 72% |
| LDN-193189 | 92% | 106% | 20% | 97% | 44% | 5% |
| LDN-212854 | 105% | 103% | 100% | 102% | 100% | 6% |
| K02288 | 103% | 107% | 115% | 106% | 110% | 35% |
| 11 | 95% | 98% | 91% | 99% | 89% | 82% |
| 12 | 96% | 103% | 93% | 97% | 88% | 89% |
| 13 | 97% | 100% | 95% | 101% | 98% | 82% |
| 14 | 96% | 110% | 99% | 99% | 93% | 10% |
| 15 | 95% | 103% | 103% | 96% | 96% | 23% |
| 16 | 92% | 104% | 108% | 101% | 93% | 25% |
| 17 | 95% | 99% | 68% | 99% | 80% | 5% |
| 18 | 98% | 101% | 106% | 103% | 100% | 9% |
| 19 | 100% | 102% | 91% | 103% | 95% | 6% |
| 20 | 106% | 102% | 17% | 108% | 104% | 5% |
| 21 | 94% | 105% | 64% | 98% | 82% | 5% |
| 22 | 96% | 104% | 21% | 101% | 81% | 5% |
| 23 | 95% | 101% | 35% | 98% | 86% | 5% |
| 24 | 95% | 105% | 86% | 101% | 86% | 9% |
| 25 | 92% | 103% | 95% | 101% | 91% | 22% |
| 26 | 96% | 103% | 62% | 103% | 80% | 5% |
| 27 | 95% | 103% | 97% | 101% | 98% | 19% |
| 10 | 91% | 100% | 88% | 92% | 78% | 77% |
| 28 | 92% | 102% | 5% | 99% | 98% | 5% |
| 29 | 91% | 104% | 108% | 108% | 84% | 24% |
| 30 | 92% | 104% | 11% | 104% | 92% | 5% |
| 31 | 96% | 103% | 117% | 105% | 95% | 16% |
| 32 | 92% | 101% | 65% | 99% | 65% | 5% |
| 33 | 95% | 100% | 23% | 101% | 92% | 5% |

### Example 7

### Kinome Profiling

The kinome-wide selectivity of compounds 10 and 15 was determined via enzymatic kinase profiling of approximately 200 kinases. The kinome-wide selectivity was determined following the methods previously reported by Mohedas et al., ACS Chem Biol 2013, 8, 1291-1302 and Sanvitale et al., PLoS One 2013, 8, e62721. The results of kinome profiling are shown in Tables 4 and 5.

TABLE 4
Inhibitory Activity of compound 10 at 100 nM and 1 μM.

10

| Kinase | 100 nM | 1 μM |
|---|---|---|
| ALK2 | 67 | 99 |
| TNIK | 71 | 98 |
| RIPK2 | 71 | 97 |
| ABL1 | 56 | 93 |
| MAP4K4 | 34 | 92 |
| MAP4K5 | 43 | 86 |
| LCK | 21 | 65 |
| PDGFR-BETA | 17 | 64 |
| ARG | 17 | 62 |
| MAP4K2 | 16 | 61 |
| ALK6 | 11 | 60 |
| PRKD2 | 16 | 57 |

TABLE 5
Inhibitory activity of compounds 15 and 10 at 100 nM and 1 μM for 194
kinases representing a wide sampling of the human kinome.

| | |
|---|---|
| 15 | 10 |

| Kinase | 100 nM | 1 μM | 100 nM | 1 μM |
|---|---|---|---|---|
| BRK | 43 | 92 | 7 | 36 |
| MAP4K4 | 77 | 90 | 34 | 92 |
| LCK | 65 | 86 | 21 | 65 |
| DDR2 | 32 | 86 | −1 | 20 |
| ABL1 | 77 | 84 | 56 | 93 |
| LYNA | 41 | 84 | 3 | 29 |
| LYNB | 35 | 82 | 8 | 30 |
| YES | 41 | 82 | 11 | 47 |
| HCK | 41 | 81 | 3 | 15 |
| ARG (ABL2) | 60 | 81 | 17 | 62 |
| SRC | 40 | 81 | 8 | 23 |
| FYN | 40 | 79 | 8 | 36 |
| PDGFRβ | 47 | 77 | 17 | 64 |
| MAP4K2 | 36 | 77 | 16 | 61 |
| MER | 30 | 76 | 8 | 36 |
| PDGFRα | 39 | 76 | 9 | 40 |
| FGR | 36 | 76 | 9 | 36 |
| TYRO3 | 23 | 75 | 4 | 21 |
| LOK | 32 | 73 | 9 | 47 |
| EPHB2 | 22 | 70 | 5 | 15 |
| TXK | 21 | 66 | 12 | 14 |
| PTK5 | 17 | 66 | 1 | 11 |
| FMS | 23 | 65 | 5 | 33 |
| BLK | 16 | 64 | 1 | 12 |
| LTK | 13 | 60 | 4 | 10 |
| LRRK2-G2019S | 16 | 57 | 4 | 11 |
| PRKD2 | 12 | 54 | 16 | 57 |
| PRKD1 | 11 | 50 | 4 | 23 |
| MRCK-α | 6 | 50 | −1 | 2 |
| MARK3 | 4 | 48 | 0 | 3 |
| EPH-A4 | −1 | 45 | 7 | 5 |
| EPHB4 | 7 | 45 | 5 | 8 |
| P38β | 10 | 44 | 1 | 11 |
| TNK2 | 6 | 44 | 6 | 35 |
| MARK4 | 5 | 40 | 1 | 2 |
| PRKD3 | 7 | 38 | 2 | 21 |
| EGFR | 8 | 37 | 3 | 16 |
| ALK | 6 | 36 | 2 | 7 |
| CSK | 10 | 36 | 4 | 5 |
| SRMS | 11 | 33 | 0 | 1 |
| MARK1 | 3 | 31 | 0 | 2 |
| EPHA3 | 8 | 31 | 2 | 3 |
| CK1-γ3 | 7 | 30 | 1 | 10 |
| BMX | 5 | 30 | 7 | 11 |
| ERBB4 | 9 | 29 | 4 | 21 |
| BRAF | 8 | 27 | 0 | 1 |
| TEC | 5 | 24 | 0 | 0 |
| TBK1 | 6 | 23 | 1 | 4 |
| KDR | 6 | 22 | 0 | 3 |
| KIT | 5 | 22 | 5 | 12 |
| MRCK-β | 3 | 22 | 0 | 0 |
| MET | 7 | 21 | −1 | 2 |
| IKK-ε | 1 | 20 | 2 | 2 |
| BTK | 3 | 20 | 1 | −2 |
| PAR-1Bα | 1 | 19 | 2 | 4 |
| CK1α | 5 | 18 | 2 | 7 |
| TTK | 13 | 18 | 3 | 4 |
| CK1-γ1 | 4 | 17 | 1 | 6 |
| RON | 3 | 17 | 2 | 6 |
| TNK1 | 2 | 16 | 5 | 2 |
| PYK2 | 10 | 14 | 1 | 5 |
| CK1-γ2 | 3 | 12 | 1 | 4 |
| MST1 | 3 | 10 | 3 | 3 |
| P38α | 4 | 9 | −2 | 1 |
| FGFR1 | 3 | 8 | 5 | 5 |
| RET | 8 | 8 | 2 | 2 |
| INSR | 4 | 8 | 3 | 3 |
| AURORA-B | 3 | 8 | 2 | 3 |
| ARK5 | 2 | 8 | 1 | 1 |
| CHEK2 | 2 | 7 | 1 | 8 |
| ROS | 1 | 7 | 2 | 2 |
| MNK2 | 1 | 7 | 6 | 6 |
| EPHB3 | 5 | 7 | 1 | 1 |
| AURORA-C | 3 | 7 | 1 | 1 |
| PI3-K-δ | 3 | 7 | 8 | 7 |
| MEK1 | 3 | 6 | 0 | 2 |
| NEK9 | 3 | 6 | 1 | 1 |
| CDK6/cyclinD3 | 3 | 6 | 3 | 1 |
| PAK1 | 0 | 6 | 2 | 3 |
| PKC-α | 1 | 6 | 2 | 3 |
| MAPK1 | 3 | 6 | 1 | 1 |
| TIE2 | 1 | 6 | 1 | 0 |
| FGFR3 | 2 | 6 | 0 | 0 |
| FER | 0 | 6 | 3 | 3 |
| CHEK1 | 2 | 6 | 4 | −3 |
| PAK5 | 9 | 6 | 1 | 1 |
| DYRK1A | 2 | 5 | 3 | 5 |
| FGFR2 | 2 | 5 | −1 | 0 |
| FLT-1 | 3 | 5 | 8 | 16 |
| MKNK1 | 3 | 5 | 3 | 5 |
| TSSK1 | 1 | 5 | 2 | 2 |
| MUSK | 1 | 5 | 2 | 2 |
| TRKA | 5 | 5 | 2 | 1 |
| FLT-3 | 1 | 5 | 3 | 18 |
| AMP-A1B1G1 | 0 | 5 | 5 | 1 |
| ERB-B2 | 0 | 5 | 0 | 3 |
| RSK1 | 1 | 5 | 1 | 1 |
| PHKγ1 | 3 | 4 | 3 | 3 |
| MST2 | 3 | 4 | 0 | 1 |
| RSK2 | 0 | 4 | 1 | 1 |
| PKC-γ | 1 | 4 | 3 | 5 |
| EPH-A2 | −3 | 4 | −2 | 1 |
| PRKG1 | 0 | 4 | 3 | 2 |
| FLT-4 | 1 | 4 | 3 | 9 |
| PI3-K-α | 4 | 4 | 5 | 7 |
| IGF1R | 2 | 4 | 0 | 0 |
| DYRK1B | −1 | 4 | 2 | 5 |
| FES | 3 | 4 | 7 | 9 |
| NEK6 | 0 | 4 | 1 | 0 |
| PAK6 | 5 | 3 | 1 | 5 |
| AURORA-A | 2 | 3 | 2 | 3 |
| DCAMKL2 | −1 | 3 | 1 | 0 |
| JAK3 | 0 | 3 | 3 | 3 |
| SGK3 | −8 | 3 | 3 | 11 |
| CDK4/cyclinD | 1 | 3 | −3 | 2 |
| TRKB | 2 | 3 | 3 | 3 |
| PDK1 | 3 | 3 | 3 | 6 |
| PHKγ2 | 2 | 3 | 1 | 0 |
| IKK-β | 1 | 3 | 2 | 2 |
| SGK2 | 2 | 3 | 1 | −1 |
| JNK2 | 2 | 3 | 0 | 2 |
| CAMK2δ | 1 | 3 | 1 | 1 |
| TRKC | 3 | 3 | 4 | 4 |
| IRR | 3 | 3 | 2 | 1 |
| RSK3 | 2 | 3 | 3 | 3 |
| NEK2 | 4 | 2 | 3 | 2 |
| AKT2 | 2 | 2 | −1 | −1 |
| HIPK1 | 1 | 2 | 1 | 1 |
| BRSK2 | 1 | 2 | 2 | 1 |
| AKT1 | 0 | 2 | 0 | 0 |
| AKT3 | 0 | 2 | 1 | 1 |
| CDK2/cyclinE | 1 | 2 | −1 | 3 |
| PKA | 1 | 2 | 1 | 3 |
| ROCK2 | −1 | 2 | 4 | 3 |
| CDK2/cyclinA | 1 | 2 | 4 | 1 |
| ITK | 0 | 2 | 0 | −1 |
| NEK7 | 9 | 2 | 4 | 3 |
| IRAK4 | 1 | 2 | 0 | 1 |
| RSK4 | 1 | 2 | 1 | 1 |
| HIPK4 | 1 | 2 | 5 | 8 |
| SGK1 | 3 | 1 | 2 | 1 |
| PAK3 | 1 | 1 | 2 | 2 |
| PLK1 | 3 | 1 | 4 | 3 |
| NEK1 | 1 | 1 | 2 | 1 |
| P38γ | 2 | 1 | −2 | 1 |
| PRAK | 1 | 1 | 0 | 3 |
| PKC-θ | 1 | 1 | 1 | 2 |
| PI4-K-β | 1 | 1 | −10 | −15 |
| PASK | 2 | 1 | 3 | 4 |
| ZAP70 | 1 | 1 | 4 | 5 |
| MAPKAPK3 | 1 | 1 | −1 | 0 |
| PKC-1 | 1 | 1 | 12 | 3 |
| TSSK2 | 1 | 1 | 4 | 4 |
| PRKG2 | 0 | 1 | 0 | 4 |
| PAK2 | 0 | 1 | 0 | 1 |
| P38δ | 2 | 1 | 1 | −1 |
| JAK1 | 1 | 0 | 2 | 2 |
| GRK6 | 0 | 0 | 8 | 3 |
| MSSK1 | −2 | 0 | 2 | 2 |
| PKC-β1 | 0 | 0 | 0 | 1 |
| PIM-2 | 0 | 0 | 0 | 0 |
| P70S6KB1 | 0 | 0 | 1 | 1 |
| BRSK1 | 0 | 0 | 1 | 1 |
| DAPK1 | 1 | 0 | −1 | −1 |
| CLK3 | 0 | 0 | 9 | 12 |
| MAPK3 | 1 | 0 | 1 | 2 |
| JAK2 | 0 | 0 | 5 | 4 |
| CDK5/p35 | 0 | 0 | 2 | 2 |
| PRKX | 1 | 0 | 0 | 1 |
| MSK1 | 0 | 0 | 1 | 0 |
| DYRK2 | 2 | 0 | 2 | 1 |
| CDK3/cyclinE | 0 | 0 | 2 | 2 |
| ROCK1 | 0 | 0 | 1 | 0 |
| TYK2 | 0 | 0 | 2 | 1 |
| GRK7 | 2 | 0 | 3 | 4 |
| FGFR4 | 1 | 0 | 1 | 2 |
| CDK1/cyclinB | −1 | 0 | 2 | 3 |
| GSK3α | 1 | 0 | 1 | 1 |
| SRPK1 | 1 | −1 | 2 | 2 |
| GSK3β | 0 | −1 | 2 | 4 |
| AXL | −4 | −1 | 1 | 7 |
| CAMK2α | 1 | −1 | −1 | −1 |
| CAMK4 | 21 | −1 | 0 | −1 |
| MSK2 | 1 | −1 | 0 | 1 |
| CAMK1δ | 0 | −1 | 1 | 2 |
| PKC-η | 5 | −1 | 5 | 18 |
| PIM-1 | 0 | −1 | 1 | 0 |
| CLK2 | 1 | −1 | 2 | 2 |
| PIM3 | 1 | −1 | 3 | 4 |
| IKK-α | −1 | −2 | 3 | 4 |
| MAPKAPK2 | 0 | −4 | 4 | 4 |
| SYK | 1 | −4 | 0 | −1 |
| SPHK2 | 6 | −6 | 13 | 5 |
| SPHK1 | 2 | −12 | 0 | 0 |

### Example 8

### Comparison of Compounds Across Multiple Assays

Certain compounds of the invention were compared across multiple assays including thermal shift kinase assay, ligand induced transcriptional assay, and constitutively active ALK1-5 transcriptional activity. Tables 6 and 7 highlight the results of these assays. The results demonstrate increased selectivity for ALK2 for compound 10 albeit with a reduction in potency.

TABLE 6
Results of thermal shift kinase assays with certain compounds of invention
ΔT ° C.

| | ALK2 | ALK5 | Diff. |
|---|---|---|---|
| 15 | 15.1 | 13.9 | 1.2 |
| 26 | 14.1 | 10.4 | 3.7 |
| 10 | 13.7 | 9.7 | 4.0 |

TABLE 7
Results of ligand induced transcriptional assay and Cell
based assay for certain compounds of the invention.

| | IC50 (nM) | | | | | | | | |
|---|---|---|---|---|---|---|---|---|---|
| | Ligand Induced | | | Cell Based Assay | | | | | |
| | BMP6 | TGFb | Ratio | caALK1 | caALK2 | caALK3 | caALK4 | caALK5 | Ratio5/2 |
| 15 | 1 | 58 | 41 | 24 | 5 | 8 | 25 | 23 | 5 |
| 26 | 15 | 952 | 65 | 202 | 43 | 105 | 427 | 215 | 5 |
| 10 | 67 | 14,650 | 219 | 778 | 186 | 382 | 5,535 | 4,178 | 23 |

### INCORPORATION OF REFERENCE

All publications and patents cited herein are hereby incorporated by reference in their entirety as if each individual publication or patent was specifically and individually indicated to be incorporated by reference. In case of conflict, the present application, including any definitions herein, will control

### Equivalents

While specific embodiments of the subject invention have been discussed, the above specification is illustrative and not restrictive. Many variations of the invention will become apparent to those skilled in the art upon review of this specification and the claims below. The full scope of the invention should be determned by reference to the claims, along with their full scope of equivalents, and the specification, along with such variations.

We claim:

1. A compound having a structure of Formula I or a pharmaceutically acceptable salt, ester, or prodrug thereof: 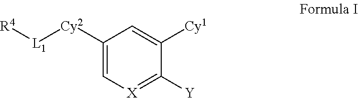
wherein
X is N;
Y is independently selected from hydrogen, cyano, carboxyl, amino, monoalkylamino, dialkylamino, halo, alkyl, or alkoxy;
Cy¹ is selected from substituted or unsubstituted aryl and heteroaryl;
Cy² is a phenyl ring substituted with at least one non-protium (¹H) substituent or a substituted or unsubstituted heteroaryl ring;
L₁ is absent or selected from substituted or unsubstituted alkyl and heteroalkyl;
R⁴ is selected from 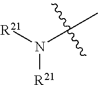
and a nitrogen-containing heterocyclyl or heteroaryl ring; and
R²¹, independently for each occurrence, is selected from H and substituted or unsubstituted alkyl, aralkyl, cycloalkyl, beterocyclyl, aryl, beteroaryl, heteroaralkyl, cycloalkylalkyl, heterocyclylalkyl, acyl sulfonyl, sulfamoyl, or sulfonamide.

2. The compound of claim 1, wherein R⁴ is 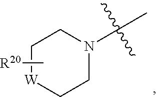
wherein
W is C(R²¹)₂, O, or NR²¹; and
R²⁰ is absent or represents from 1-6 substituents on the ring to which it is attached, independently selected from substituted or unsubstituted alkyl, aralkyl, cycloalkyl, heterocyclyl, aryl, heteroaryl, heteroaralkyl, cycloalkylalkyl, heterocyclylalkyl, aryl, sulfonyl, sulfoxido, sulfamoyl, and sulfonamido.

3. The compound of claim 2, wherein W is NR²¹.

4. The compound of claim 2 or 3, wherein R²⁰ is absent.

5. The compound of any preceding claim, wherein R²¹ is H.

6. The compound of any preceding claim, wherein Cy¹ is an aryl group substituted by 1 to 5 C₁-C₆ alkoxy groups.

7. The compound of claim 6, wherein Cy¹ is substituted by alkoxy groups in the 3-, 4- and 5-positions relative to the bond to the central pyridine ring.

8. A compound of any preceding claim, wherein Cy² is a substituted or unsubstituted nitrogen-containing heteroaryl group selected from pyridine, pyrazine, pyrimidine, oxazole, thiazole, and thiadiazole, e.g., selected from substituted or unsubstituted:

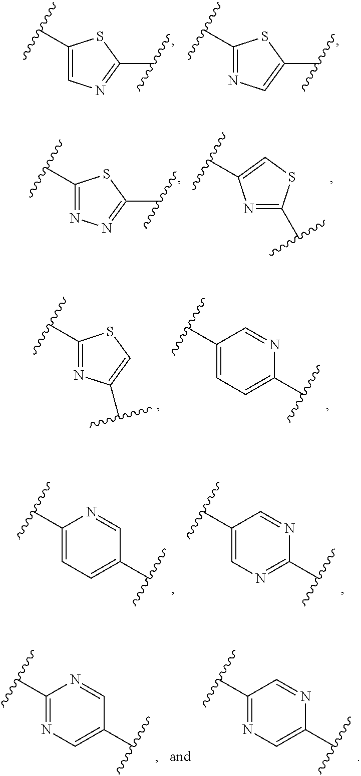

9. A compound of any preceding claim, wherein when Cy² is substituted, the substituent is selected from deuterium, halogen (preferably fluoro or chloro), hydroxy, cyano, lower alkyl (preferably methyl or ethyl, most preferably methyl), or lower alkoxy (preferably methoxy).

10. The compound of any one of claims 1-7, wherein Cy² is a phenyl ring.

11. A compound of claim 10, wherein the non-protium substituent is halogen (preferably fluoro or chloro) or cyano, or is positioned ortho to L₁, or both.

12. The compound of claim 8 or 9, wherein Cy² is a 6-membered aryl or heteroaryl ring and L₁ is disposed on the para-position of Cy² relative to the central pyridine ring.

13. The compound of any preceding claim, wherein L¹ is absent.

14. A compound of any one of claims 1-12, wherein L₁ has a structure 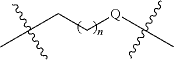
wherein
Q is selected from CR¹⁰R¹¹, NR¹², O, S, S(O), and SO₂; and
R¹⁰ and R¹¹, independently for each occurrence, are selected from H and substituted or unsubstituted alkyl, cycloalkyl, heterocyclyl, cycloalkylalkyl, heterocyclylalkyl, amino, acylamino, carbamate, amido, amidino, cyano, sulfonyl, sulfoxido, sulfamoyl, or sulfonamido;
R¹² selected from H and substituted or unsubstituted alkyl, cycloalkyl, heterocyclyl, heterocyclylalkyl, amino, acylamino, carbamate, amido, amidino, sulfonyl, sulfamoyl, or sulfonamido and
n is an integer from 0-4,
wherein any CH₂ subunit of L₁ is optionally substituted with one or two lower alkyl groups, preferably one or two methyl groups.

5. A compound of any preceding claim, wherein R⁴ is 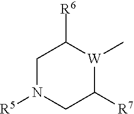
W is N, CH, or CCH₃, preferably N or CH;
R⁵ is selected from H and substituted ur unsubstituted alkyl, acyl, or ester (thereby forming a carbamate); and
R⁶ and R⁷ are each independently selected from H or alkyl, preferably from H or methyl, or R⁶ forms a one- or two-carbon (e.g., CH₂ or CH₂CH₂) bridge to the carbon atom adjacent to R⁷ and NR⁵.

16. A compound having a structure of Formula II or a pharmaceutically acceptable salt, ester, or prodrug thereof: 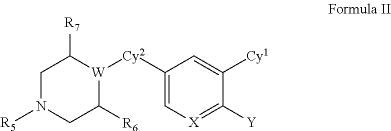
wherein
X is N;
Y is independently selected from hydrogen, cyano, carboxyl, amino, monoalkylamino, dialkylamino, halo, alkyl, or alkoxy;
Cy¹ is selected from substituted or unsubstituted aryl and heteroaryl;
Cy² is a substituted or substituted aryl or heteroaryl ring;
W is N, CH, or CCH₃, preferably N or CH;
R⁵ is selected from H and substituted or unsubstituted alkyl, acyl, or ester (thereby forming a carbamate); and
R⁶ and R⁷ are each independently selected from H or alkyl, preferably from H or methyl, or R⁶ forms a one- or two-carbon (e.g., CH₂ or CH₂CH₂) bridge to the carbon atom adjacent to R⁷ and NR⁵.

17. A compound according to claim 16, wherein R₆ and R₇ are both methyl, optionally disposed in a syn relationship to each other.

18. A compound according to claim 16, wherein R₆ represents a one-carbon bridge, thereby forming a diazanorbornane bicycle.

19. The compound of any one of claims 16-18, wherein W is N.

20. The compound of any one of claims 16-19, wherein Cy¹ is an aryl group substituted by 1 to 5 C₁-C₆alkoxy groups.

21. The compound of claim 20, wherein Cy¹ is substituted by alkoxy groups in the 3-, 4- and 5- positions relative to the bond to the central pyridine ring.

22. A compound of any one of claims 16-21, wherein Cy² is a substituted or unsubstituted nitrogen-containing heteroaryl group selected from pyridine, pyrazine, pyrimidine, oxazole, thiazole, and thiadiazole, e.g., selected from substituted or unsubstituted:

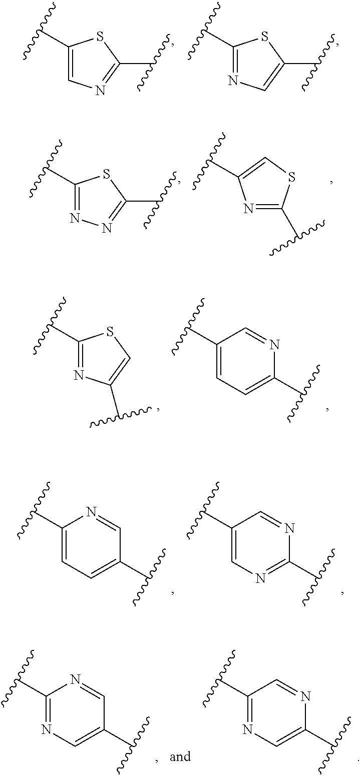

23. A compound of any one of claims 16-22, wherein when Cy² is substituted, the substituent is selected from deuterium, halogen (preferably fluoro or chloro), hydroxy, cyano, lower alkyl (preferably methyl or ethyl, most preferably methyl), or lower alkoxy (preferably methoxy).

24. The compound of any one of claims 16-21, wherein Cy² is a phenyl ring.

25. A compound of claim 24, wherein the phenyl ring has at least one non-protium substituent, wherein the non-protium substituent is optionally selected from halogen (preferably fluoro or chloro) or cyano, or is positioned ortho to W, or both.

26. The compound of claim 22 or 23, wherein Cy² is a 6-membered aryl or heteroaryl ring and W is disposed on the para-position of Cy² relative to the ring bearing X.

27. The compound of any preceding claim, wherein Y is deuterium, amino, monoalkylamino, or dialkylamino, preferably amino, monoalkylamino, or dialkylamino, most preferably amino.

28. A pharmaceutical composition comprising a compound of any preceding claim and a pharmaceutically acceptable excipient or solvent.

29. A method of inhibiting BMP-induced phosphorylation of SMAD1/5/8, comprising contacting the cell with a compound of any one of claims 1-27.

30. The method of claim 29, wherein the method treats or prevents a disease or condition in a subject that would benefit by inhibition of Bone Morphogenetic Protein (BMP) signaling.

31. The method of claim 30, wherein the disease or condition is selected from pulmonary hypertension, hereditary hemorrhagic telangiectasia syndrome, cardiac valvular malformations, cardiac structural malformations, fibrodysplasia ossificans progressiva, Juvenile familial polyposis syndrome, parathyroid disease, cancer, anemia, vascular calcification, atherosclerosis, valve calcification, renal osteodystrophy, inflammatory disorders, and infections with viruses, bacteria, fungi, tuberculosis, and parasites.

32. The method of claim 31, wherein the disease or condition is a cancer selected from breast carcinoma, prostate carcinoma, renal cell carcinoma, bone metastasis, lung metastasis, osteosarcoma, and multiple myeloma.

33. The method of claim 31, wherein the disease or condition is an inflammatory disorder such as ankylosing spondylitis.

34. A method of inducing expansion or differentiation of a cell, comprising contacting the cell with a compound of any of claims 1-27.

35. The method of claim 34, wherein the cell is selected from an embryonic stem cell and an adult stem cell.

36. The method of claim 34 or 35, wherein the cell is in vitro.

37. A method of reducing circulating levels of ApoB-100 or LDL in a subject, comprising administering an effective amount of a compound of any one of claims 1-27.

38. A method of treating hypercholesterolemia, hyped ipidemia, or hyperlipoproteinemia in a subject, comprising administering an effective amount of a compound of any one of claims 1-27.

39. The method of claim 38, wherein the hypercholesterolemia, hyperlipidemia, or hyperlipoproteinemia is congenital hypercholesterolemia, hyperlipidemia, or hyperlipoproteinernia.

40. The method of claim 39, wherein the hypercholesterolemia, hyperlipidemia, or hyperlipoproteinemia is autosomal dominant hypercholesterolemia (ADH), familial hypercholesterolemia (FH), polygenic hypercholesterolemia, familial combined hyperlipidemia (FCHL), hyperapobetalipoproteinemia, or small dense LDL syndrome (LDL phenotype B).

41. The method of claim 38, wherein the hypercholesterolemia, hyperlipidemia, or hyperlipoproteinemia is acquired hypercholesterolemia, hyperlipidemia, or hyperlipoproteinemia.

42. The method of claim 41, wherein the hypercholesterolemia, hyperlipidemia, or hyped ipoproteinemia is associated with diabetes mellitus, hyperlipidemic diet and/or sedentary lifestyle, obesity, metabolic syndrome, intrinsic or secondary liver disease, primary biliary cirrhosis or other bile stasis disorders, alcoholism, pancreatitis, nephrotic syndrome, endstage renal disease, hypothyroidism, iatrogenesis due to administration of thiazides, beta-blockers, retinoids, highly active antiretroviral agents, estrogen, progestins, or glucocorticoids.

43. A method of treating diseases, disorders, or syndromes associated with defects in lipid absorption or metabolism or caused by hyperlipidemia in a subject, comprising administering an effective amount of a compound of any one of claims 1-27.

44. A method of reducing secondary cardiovascular events arising from coronary, cerebral, or peripheral vascular disease in a subject, comprising administering an effective amount of a compound of any one of claims 1-27.

45. A method of preventing cardiovascular disease in a subject with elevated markers of cardiovascular risk, comprising administering an effective amount of a compound of any one of claims 1-27.
